(19) United States
(12) Patent Application Publication
(10) Pub. No.: US 2022/0344011 A1
(43) Pub. Date: Oct. 27, 2022

(54) SYSTEM AND METHODS FOR ENHANCED MANAGEMENT OF PATIENT CARE AND COMMUNICATION

(71) Applicant: Jaan Health, Inc., Cincinnati, OH (US)

(72) Inventors: Nabeel Iqbal Kaukab, Bellevue, WA (US); Toufique Harun, Albertson, NY (US)

(73) Assignee: Jaan Health, Inc., Cincinnati, OH (US)

(21) Appl. No.: 17/663,211

(22) Filed: May 12, 2022

Related U.S. Application Data
(63) Continuation of application No. 16/509,484, filed on Jul. 11, 2019, now abandoned.
Application No. 16/509,484 is a continuation of application No. 14/845,553, filed on Sep. 4, 2015, now abandoned.
Application No. 14/845,553 is a continuation-in-part of application No. 14/566,706, filed on Dec. 10, 2014, now abandoned.
(60) Provisional application No. 61/913,924, filed on Dec. 10, 2013.

(51) Int. Cl. G16H 10/60; H04L 51/066; G16H 10/20; G16H 50/30; G16H 40/20; G16H 80/00

(52) U.S. Cl. CPC G16H 10/60; H04L 51/066; G16H 10/20; G16H 50/30; G16H 40/20; G16H 80/00; G16H 15/00

(57) ABSTRACT

A method and system for enhanced management of patient care and communication, wherein a provider may select a pre-existing template, generate a message from the pre-existing template, search for recipients based on specific criteria, determine which recipients may receive the message based on a message type selection and the specific criteria, and then send the message to said recipients.

### CROSS REFERENCE TO RELATED APPLICATIONS

This application is a continuation of U.S. patent application Ser. No. 16/509,484, filed on Jul. 11, 2019, which is a continuation of U.S. patent application Ser. No. 14/845,553, filed on Sep. 4, 2015, which is a continuation-in-part of U.S. patent application Ser. No. 14/566,706, filed on Dec. 10, 2014, which claims the benefit of U.S. Provisional Patent Application Ser. No. 61/913,924, filed on Dec. 10, 2013, the entireties of which are hereby incorporated herein by reference.

### BACKGROUND

### Field of the Invention

The present disclosure relates to systems and methods for enhanced management of patient care and communication.

### Description of the Related Art

Generally, when a patient sees a health care provider and is given a treatment plan, the only written information that a patient may receive is a prescription or a discharge plan. Any prescription information is usually transferred to medication labels, which a patient must typically consult to determine if that aspect of the treatment plan is being properly followed. If the patient has any symptoms or other issues relating to the treatment plan, a patient must contact the health care provider to report the concern, typically by phone. If the health care provider wishes to follow up on the concern or check on the progress of the treatment plan, the health care provider generally must contact the patient by phone or schedule a consultation with the patient.

### SUMMARY

A computer operable method is described for enhanced management of patient care and communication. The method may comprise sending a list of pre-existing templates; receiving a pre-existing template selection for generating a message; receiving a message type selection; sending a set of search options for determining recipients contained in a patient database; identifying a set of recipients in the patient database that match the search parameters upon the receipt of said search parameters, including whether the recipient is permitted to receive the message based on the message type selection; and sending the message to the set of recipients based on the message type selection.

A system is described for enhanced management of patient care and communication. They system may comprise a database component containing patient records; and a notification/messaging component for handling messages, wherein the notification/messaging system is capable of: generating a message from a pre-existing template; generating a set of search options for determining recipients contained in the patient records; requesting the database component to identify a set of recipients based on a set of search parameters and a message type selection; receiving the set of recipients; formatting the message based on the message type selection; and sending the formatted message to the set of recipients.

### BRIEF DESCRIPTION OF THE DRAWINGS

FIG. 1A is a block diagram of an exemplary system for performing the embodiments disclosed herein.

FIG. 1B is a block diagram of an exemplary system for performing the embodiments disclosed herein.

FIG. 2 illustrates an embodiment of a methodology operable by a computing device for accessing a User Home Page.

FIG. 3 illustrates an embodiment of a methodology operable by a computing device for obtaining User Registration information.

FIG. 4 illustrates an embodiment of a methodology operable by a computing device for selecting or adding a Family Member.

FIG. 5 illustrates an embodiment of a methodology operable by a computing device for selecting or adding a provider.

FIG. 6 illustrates an embodiment of a methodology operable by a computing device for selecting or adding a medication.

FIG. 7 illustrates an embodiment of a methodology operable by a computing device for accessing or editing information from a User Home Page.

FIG. 8 illustrates an embodiment of a methodology operable by a computing device for accessing or editing information from a General Settings Page.

FIG. 9 illustrates an embodiment of a methodology operable by a computing device for accessing medication information or editing medication usage information.

FIG. 10 illustrates an embodiment of a methodology operable by a computing device for accessing or editing symptom status.

FIG. 11 illustrates an embodiment of a methodology operable by a computing device for accessing or editing providers.

FIG. 12 illustrates an embodiment of a methodology operable by a computing device for notifying a user of a need to take a medication.

FIG. 13 illustrates an embodiment of a methodology operable by a computing device for providing an overview of user status to a provider.

FIG. 14 illustrates an embodiment of a methodology operable by a computing device for providing an overview of particular user information to a provider.

FIG. 15 illustrates an embodiment of a methodology operable by a computing device for calculating severity levels and scores in relation to medication or Symptom Status information.

FIG. 16A illustrates an example of a Login/Signup Page.

FIG. 16B illustrates an example of a Login Page.

FIG. 16C illustrates an example of a User Registration Page.

FIG. 16D illustrates an example of a Provider Selection Page.

FIG. 16E illustrates an example of an Add Provider Page.

FIG. 16F illustrates an example of an Add Family Member Page.

FIG. 16G illustrates an example of a Medication Selection Page.

FIG. 16H illustrates an example of a Medication Usage Page.

FIG. 16I illustrates an example of a User Home Page.

FIG. 16J illustrates an example of a Medication Information Page.

FIG. 16K illustrates an example of a General Settings Page.

FIG. 16L illustrates an example of a My Providers Page.

FIG. 16M illustrates another example of a Medication Information Page.

FIG. 16N illustrates an example of a My Family Page.

FIG. 16O illustrates an example of a Help Page.

FIG. 16P illustrates an example of a History Page.

FIGS. 17A-F illustrate examples of a Symptom Status Page.

FIGS. 18A-B illustrate additional examples of a Symptom Status Page.

FIG. 19A illustrates an example of a Reminder Notice.

FIG. 191B illustrates another example of a Reminder Notice.

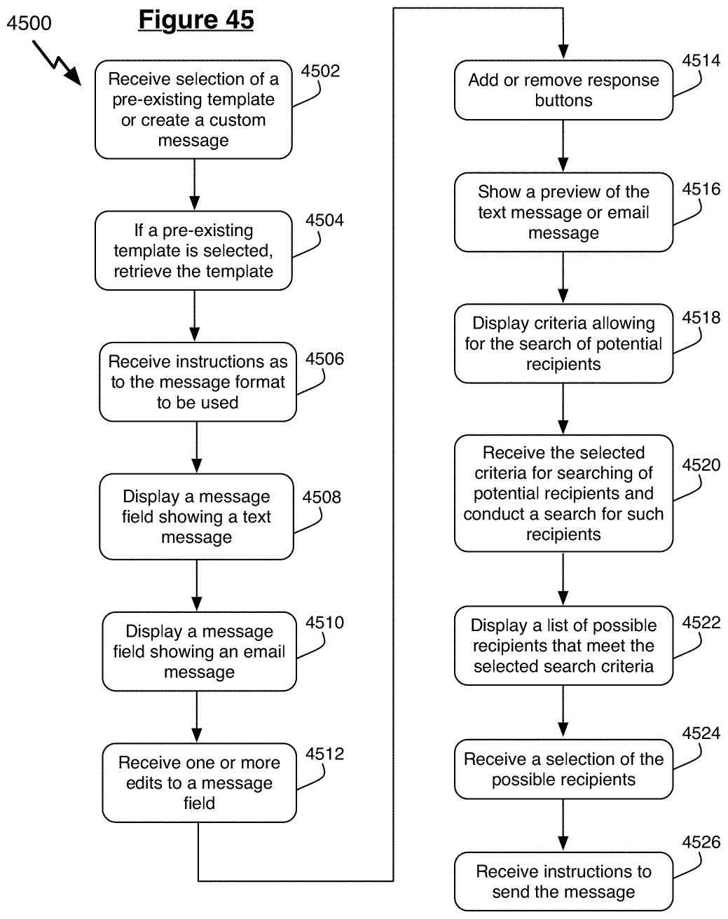

FIG. 20 illustrates an example of a Profile Page.

FIG. 21 illustrates an example of a Provider Dashboard Page.

FIG. 22A illustrates an example of a Patient Information Page.

FIG. 22B illustrates an example of a Patient Medication History Page.

FIG. 22C illustrates an example of a Patient Contact Menu Page.

FIG. 22D illustrates an example of a Patient Adherence Graph Page.

FIG. 22E illustrates an example of a Patient Side Effects Graph Page.

FIG. 23 illustrates an embodiment of a methodology operable by a computing device.

FIG. 24 illustrates an embodiment of a methodology operable by a computing device.

FIG. 25A illustrates an example of a Prescription Entry Page.

FIG. 25B illustrates an example of a Patient Medication List Page.

FIG. 26A illustrates an example of an Action Plan Entry Page.

FIG. 26B illustrates an example of an Action Plan List Page.

FIG. 27 illustrates an example of a Patient Medication Report.

FIG. 28 illustrates an example of an Action Plan Report.

FIG. 29 illustrates another example of an Action Plan Report.

FIG. 30 illustrates another example of a Provider Dashboard Page.

FIG. 31 illustrates an example of a Provider Message Options Page.

FIG. 32 illustrates an example of a Provider Message Draft Page.

FIG. 33 illustrates an example of a Provider Appointment Reminder Page.

FIG. 34 illustrates an example of a Patient Interaction Report.

FIG. 35 illustrates an example of a Patient Dashboard Page.

FIG. 36 illustrates an example of a Patient Message Page.

FIG. 37 illustrates another example of a Patient Message Page.

FIG. 38 illustrates another example of a Medication Usage Page.

FIG. 39 illustrates an example of a Patient Survey Page.

FIGS. 40-43 illustrate an example of a Message Template Page.

FIG. 44 illustrates an example of a methodology operable by a computing device for handling messages between a patient or a patient's agent and a provider.

FIG. 45 illustrates a methodology for selecting a pre-existing template or creating a custom message.

### DETAILED DESCRIPTION

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” “computing device,” or “system.” Furthermore, aspects of the present invention may take the form of a computer program product embodied in one or more computer readable medium(s) having computer readable program code embodied thereon.

Any combination of one or more computer readable medium(s) may be utilized. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming or scripting languages, including an object oriented programming language such as Java, Ruby, Smalltalk, C++, or the like and conventional procedural programming languages, such as the “C” programming language or similar programming languages or scripting languages, such as Python, Perl, and other goal-oriented or high level programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations or block diagrams of methods, apparatus (systems), and computer program products according to embodiments of the invention. It will be understood that each block of the flowchart illustrations or block diagrams, and combinations of blocks in the flowchart illustrations or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart or block diagram block or blocks. In the following description, certain specific details of programming, software modules, user selections, network transactions, database queries, database structures, etc. are omitted to avoid obscuring the invention. Those of ordinary skill in computer sciences will comprehend many ways to implement the invention in various embodiments, the details of which can be determined using known technologies.

Furthermore, the described features, structures, or characteristics may be combined in any suitable manner in one or more embodiments. In general, the methodologies of the present invention are advantageously carried out using one or more digital processors, for example the types of microprocessors that are commonly found in servers, PCs, laptops, PDAs, tablets, smartphones and other mobile devices, and all manner of desktop or portable electronic appliances.

The methods and systems disclosed herein are related to, but not limited to, improving care for users participating in a health-monitoring service. Accordingly, any services using the methods and systems disclosed herein may use such methods and systems to allow providers (e.g., physicians, nurses, case managers, therapists, etc.) or agents (e.g., family members, caregivers, etc.) the ability to participate in monitoring and responding to health care issues recorded by a user of the service or the service on behalf of the user. The use of the terms “providers,” “agents,” and “user” are used herein only to facilitate discussion, and carry no particular significance in terms of any form of relationship between them (e.g., contractual, familial), unless otherwise indicated.

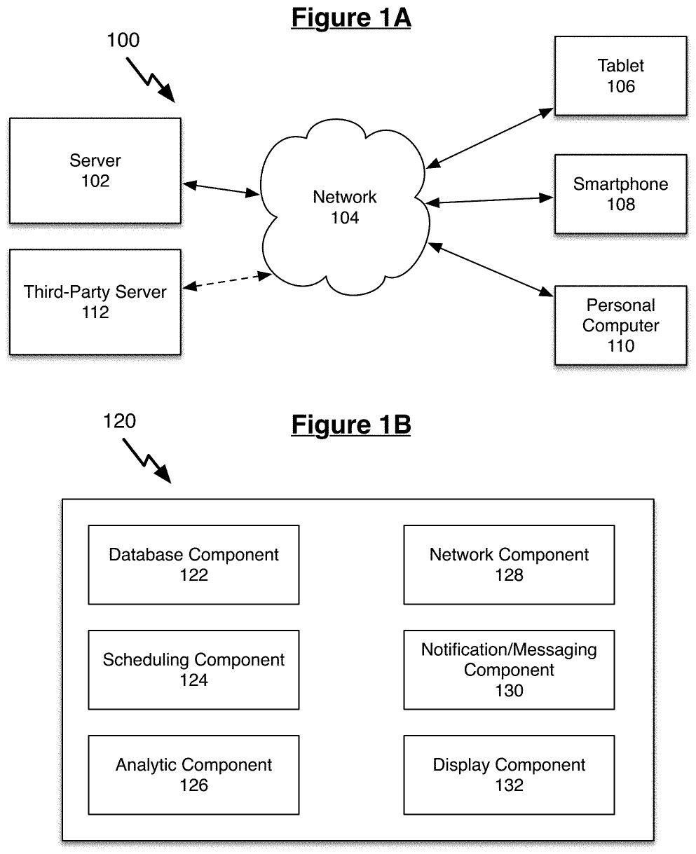

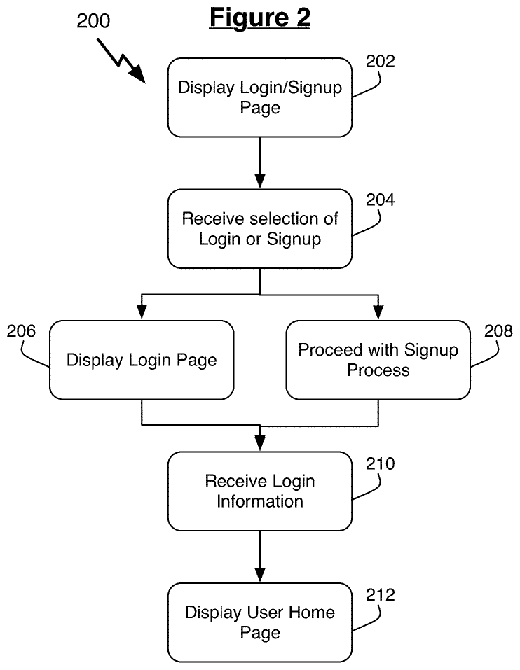

With reference to FIG. 1A, there is shown one embodiment of a system 100 that may comprise a plurality of system entities, such as a Server 102, a Network 104, a Tablet 106, a Smartphone 108, and a Personal Computer 110. Server 102 is capable of operatively communicating via Network 104 with Tablet 106, Smartphone 108, or Personal Computer 110. In addition. Tablet 106, Smartphone 108, or Personal Computer 110 may capable of operatively communicating with each other via Network 104. Network 104 may consist of a variety of different network technologies, which for example may be the Internet, a private network, a Virtual Private Network, a Wi-Fi network, a cellular data network, an interconnection of network services, or any other system which communicates using packetized communication. In related aspects, one or more of the system entities may comprise distributed components, such as a cloud computing platform, to provide the functionality of one or more of the system entities. In further related aspects, one or more of the system entities may perform the function of another system entity, such as Tablet 106, Smartphone 108, or Personal Computer 110 performing steps described for Server 102 or vice versa. Within FIG. 1A, the function of a computing device may be Server 102, Tablet 106, Smartphone 108, or Personal Computer 110, though other devices or systems may also perform as a computing device as described herein.

With reference to FIG. 1B, there is shown a system 120 that may be used to enable the methods disclosed herein. System 120 may contain a Database Component 122 for storing information, such as user records, health histories, provider-patient/agent chat transcripts, medication records, provider records, agent records, etc. User records stored by Database Component 122 may contain information for each user, such as a username, password, biometric data, a first name, a last name, a date of birth, one or more email addresses (e.g., personal, work), one or more phone numbers (e.g., home, mobile), one or more street addresses (e.g., home, work), medications, prescriptions, treatments, therapies, adherence data, symptom status data, authorized agents, authorized providers, insurance information, scheduling information, physical attributes, user preferences and defaults, preferred pharmacy, etc.

Medication records stored by Database Component 122 may contain information for each medication that may be taken by or applied to a user, such as known side effects, severity scoring relating to such side effects, brand name, active pharmaceutical ingredient, manufacturer, preferred times or frequencies of dosage, strengths of medications (e.g., 10 mg, 20 mg), different forms of medication (e.g., pill, liquid), directions for use, cautions, cost, national drug codes or similar identifiers, etc. Medication records may also further include information relating to over-the-counter supplements, such as the information typically presented on labels of vitamins or other supplements.

Provider records stored by Database Component 122 may contain information for each provider, such as a provider's first name, last name, institution, license to practice (e.g., doctor, therapist), type of practice (e.g., cardiologist, anesthesiologist), phone number, email address, street address, office hours, preferred severity scores, current or past medications, current or past treatments, current or past therapies, current or past users under provider's care, provider preferences and defaults, accepted insurance providers, user preferences and defaults, etc.

Agent records stored by Database Component 122 may contain information for each agent in a similar fashion to user records. In some embodiments, agent records may contain less information than a typical user record, such as where they are offered a limited functionality compared to a user unless they pay a fee. In other embodiments, agent records may contain more information than a typical user record, such as information relating to the legal rights to oversee any user's care (e.g., power of attorney) stored in the agent records.

System 120 may also contain a Scheduling Component 124 for scheduling events to occur, such as notices, alerts, reminders, wake-up calls, etc., or determining if an event has occurred, such as whether medication has been taken, whether symptoms have been reported, whether a procedure or provider encounter has taken place, etc.

System 120 may also contain an Analytic Component 126 for performing analysis of information, such as determining a user's adherence to a prescribed treatment or medication; determining a user's risk profile or severity score in terms of the user's medical history, reported symptoms, or pain level and specific user feedback; or other analytical functions.

System 120 may also contain a Network Component 128 for handling communications within the system or between the system and another entity, such as another system. In some embodiments, Network Component 128 may provide additional network services for the exchange of information between System 120 and a computing device outside of System 120, such as for example a Third-Party Server 112 shown in System 100 of FIG. 1A. In such embodiments, this exchange of information may be established through various means, such as an application programming interface (“API”), etc. In some embodiments, a Third-Party Server 112 may also constitute a computing device, such as where an API allows it to perform the processes and methods described herein.

System 120 may also contain a Notification/Messaging Component 130 for automating the notification or exchange of information within the system or between the system and another entity, such as another system. For example, Notification/Messaging Component 130 may, in response to information received from Scheduling Component 124, send a notification to a user that it is time to take a medication or to an agent that a user has not taken a medication within a certain period of time. As a further example, the system may notify a patient who has not reported a health status within a given period of time or on a regular basis depending upon the patient's medical history. As a further example, Notification/Messaging Component 130 may also generate messages for use with a computerized auto-responder, such as an auto-dialer, to inform a user that a provider is aware of symptoms reported by the user and that the user should continue to monitor his or her health and let the provider know of any negative changes in his or her health.

System 120 may also contain a Display Component 132 for displaying or sending instructions for displaying information, such as a User Home Page, on an electronic display. The use of the terms “Page” used herein is only to facilitate discussion, and carry no particular significance in terms of any form of displaying information, unless otherwise indicated. For example, a Page may consist of a webpage generated using HTML, XML, or other languages or the rendering of such a webpage. In addition, a Page may also allow for interactive entry of information by a user.

With respect to System 120, a computing device may provide one or more of the components described herein with respect to System 120. In addition, a computing device may only provide a portion of each component that it may contain and rely upon other devices or systems within System 120 to provide access to the remainder of each component that it does not contain. For example, System 120 may consist of one or more of the elements described with respect to System 100, each of which may act as a computing device for various or different steps with respect to the methodologies described herein.

For the descriptions below, an agent may perform in a similar manner as described with respect to a user. In addition, if a user or System 120 has authorized an agent or provider to act on the behalf of a user, that agent or provider may receive notifications, add medications or providers, or report Symptom Status as if the agent or provider was also the user. In some embodiments, when a user selects an agent, such as when accessing a My Family Page from a General Settings Page or User Home Page, a computing device may allow the authorized agent to access the user's account in the same manner as if the agent was the user. In additional embodiments, a user may be able to restrict an authorized agent or provider from performing certain actions or reviewing certain information (e.g., an agent or provider may not be authorized to provide Symptom Status information for certain medications or to see that the user is using certain medications).

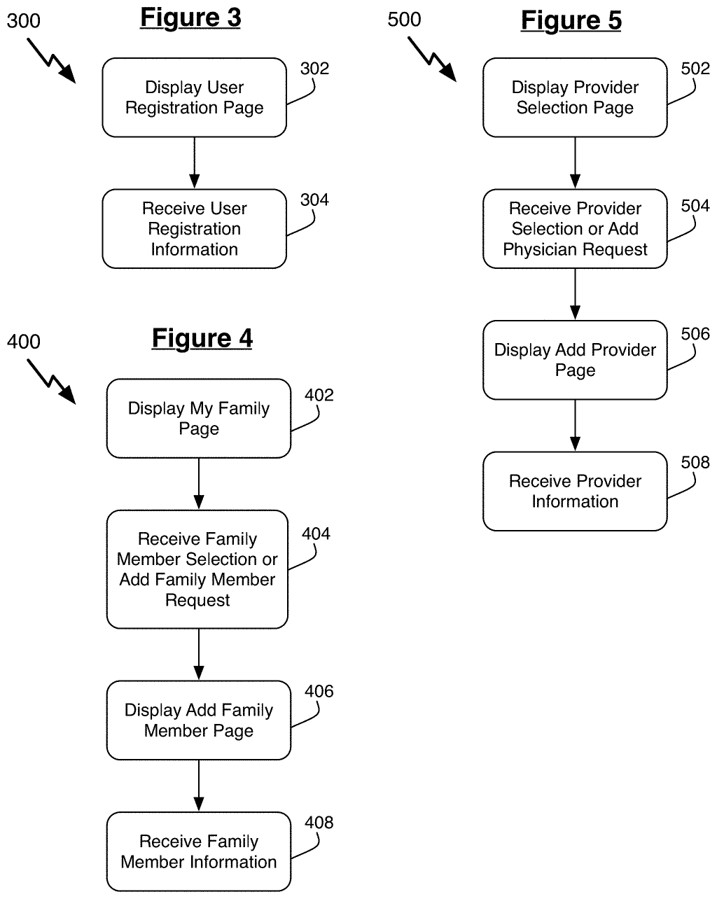

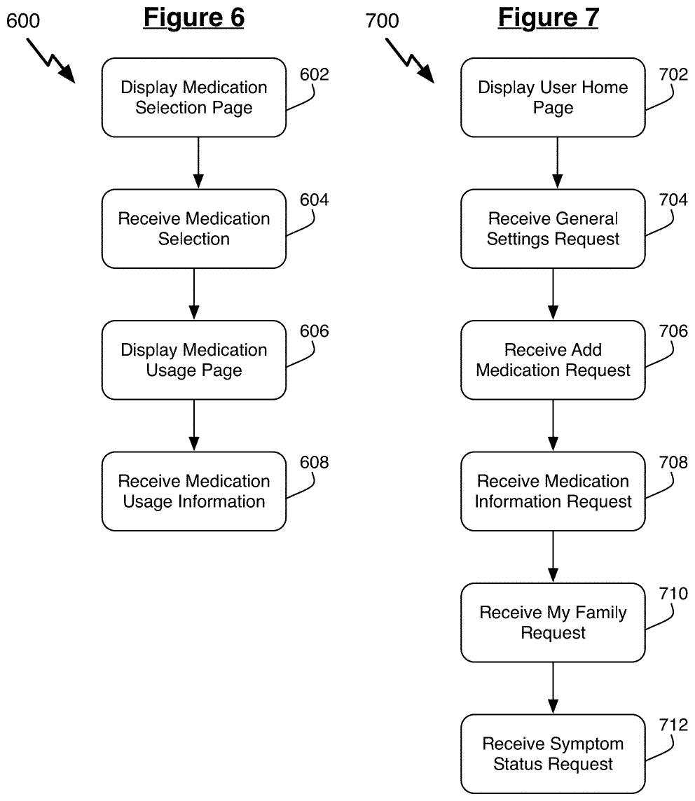

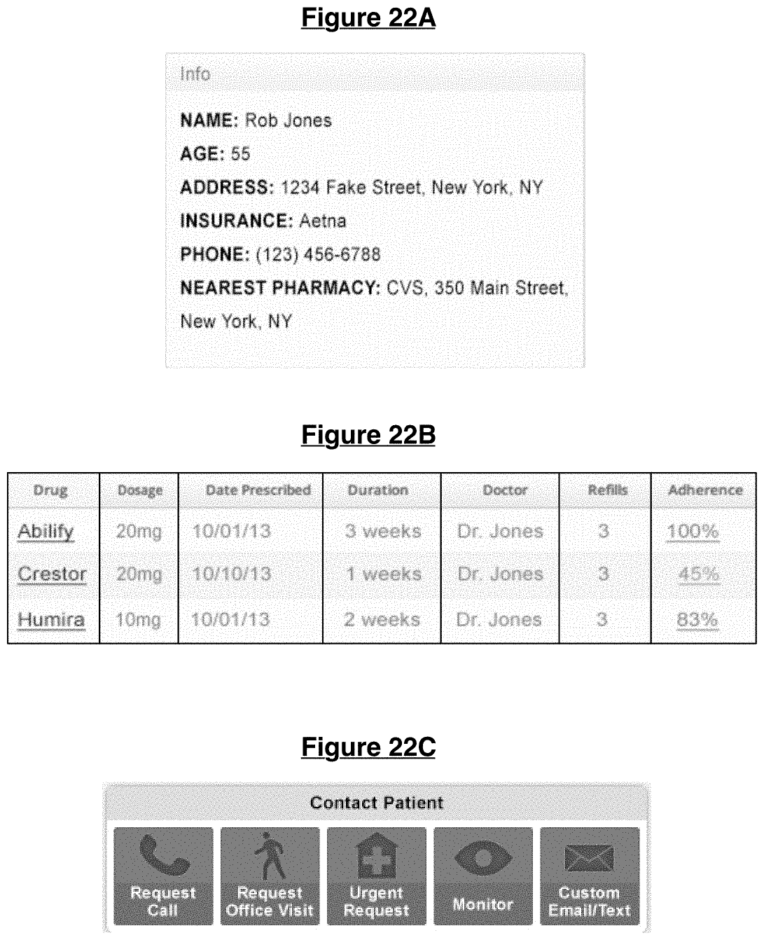

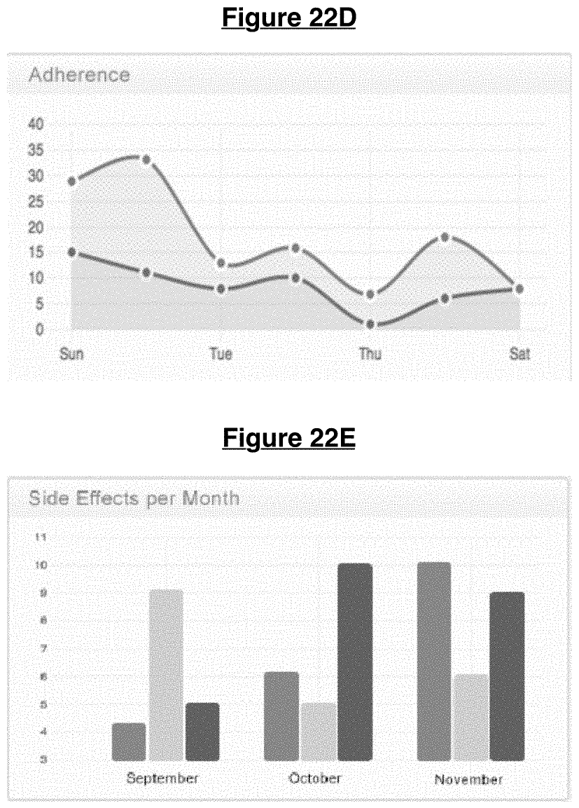

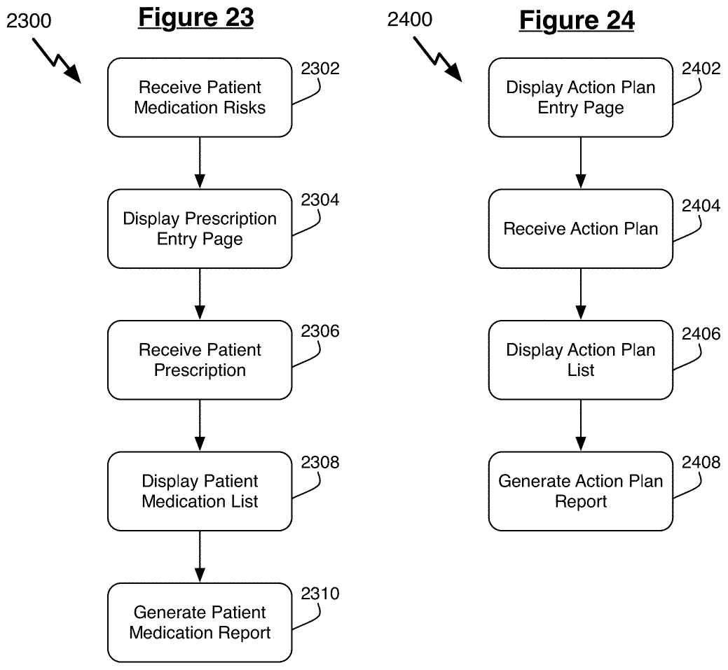

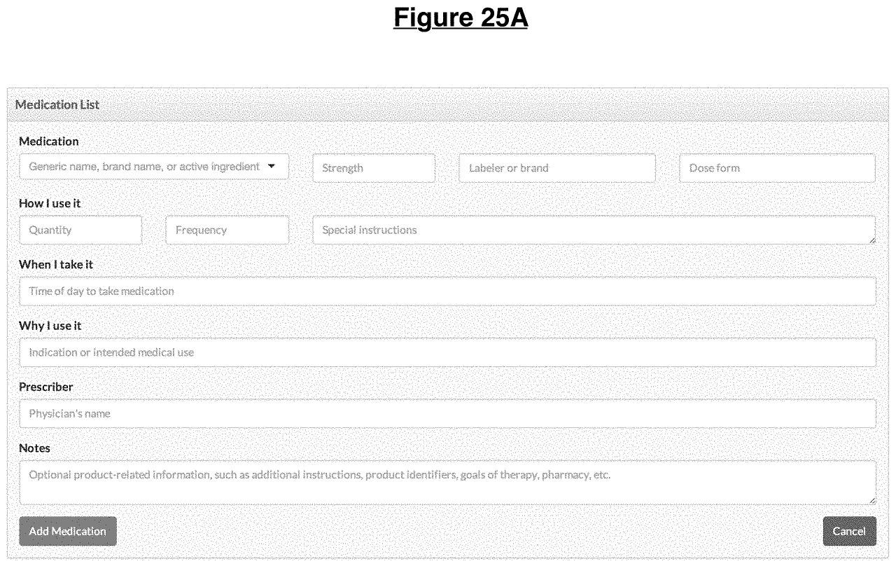

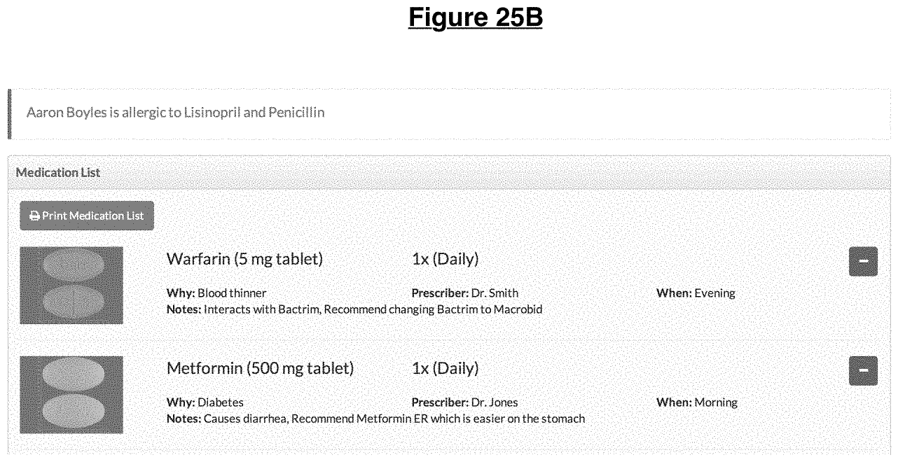

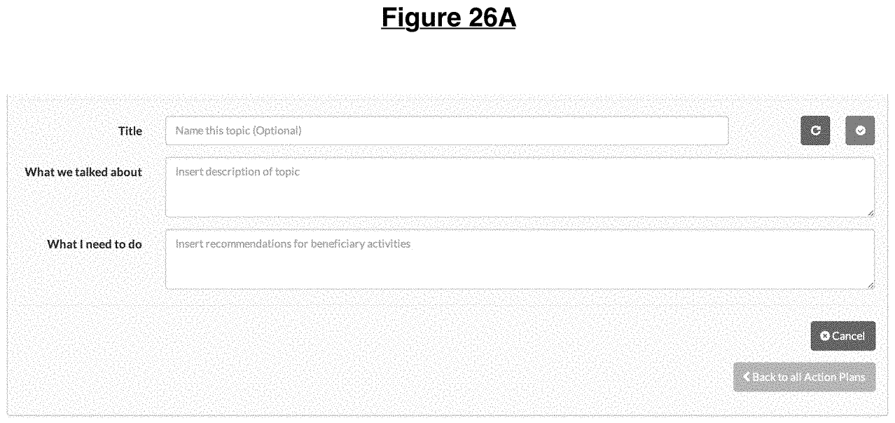

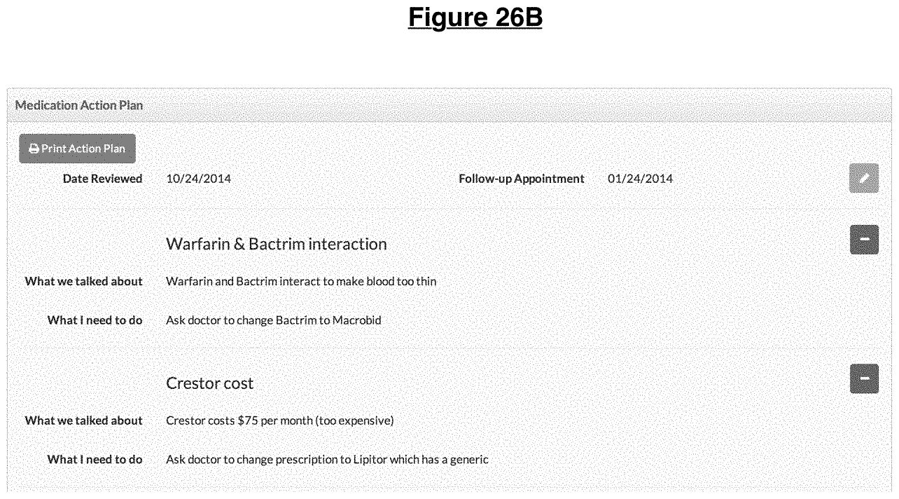

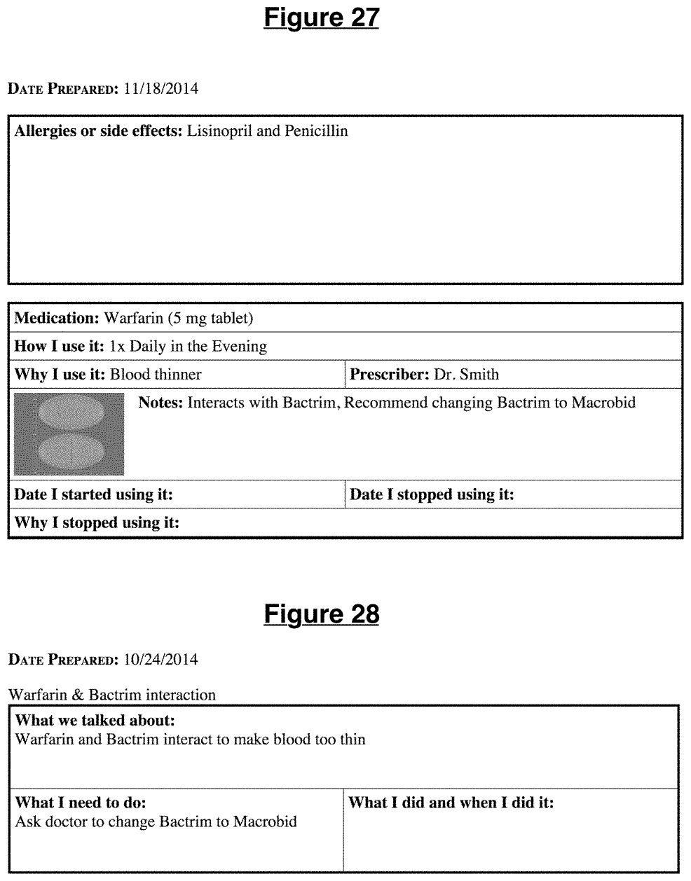

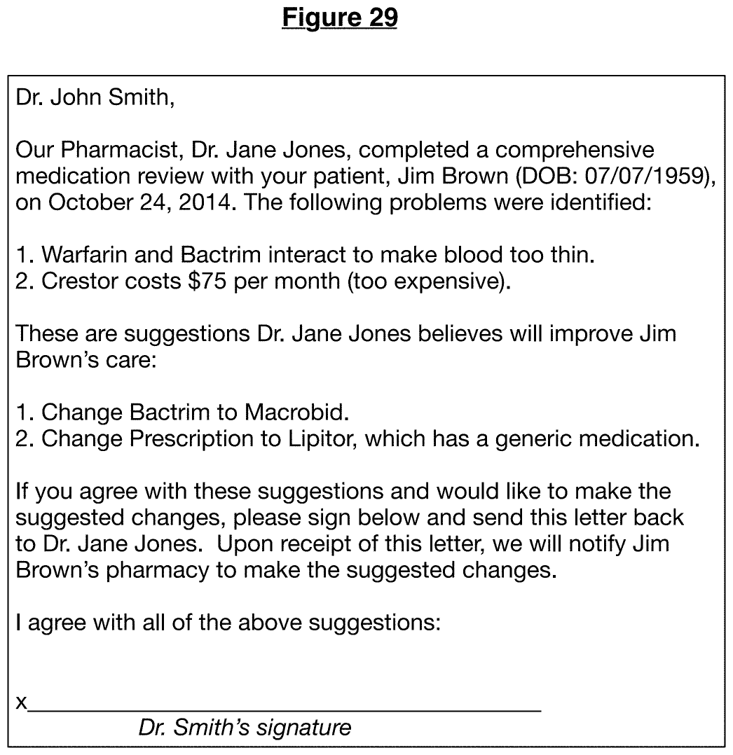

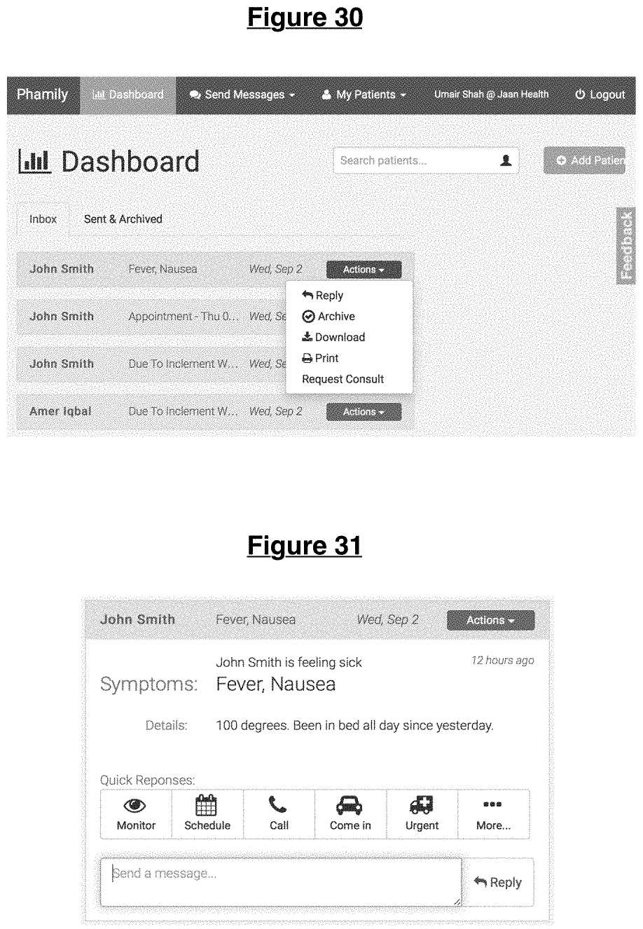

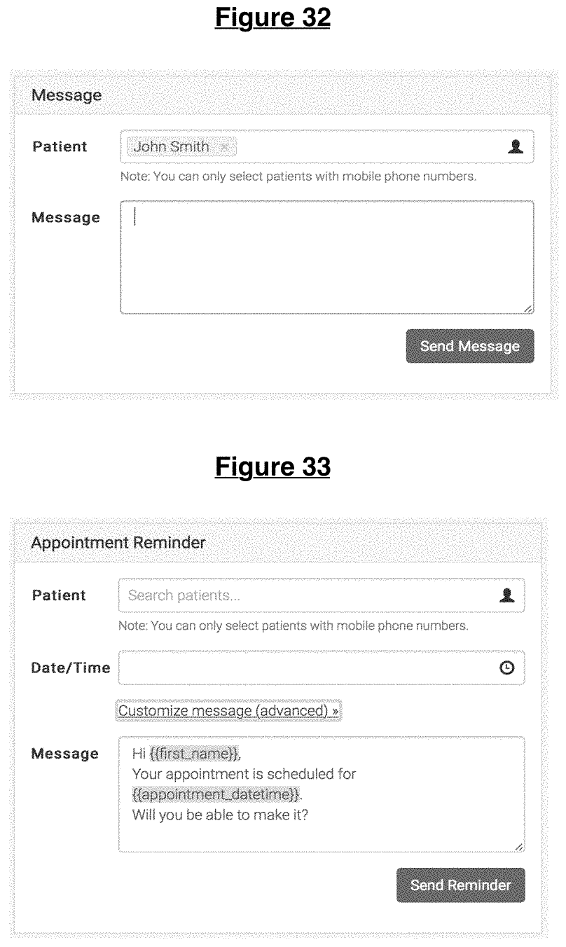

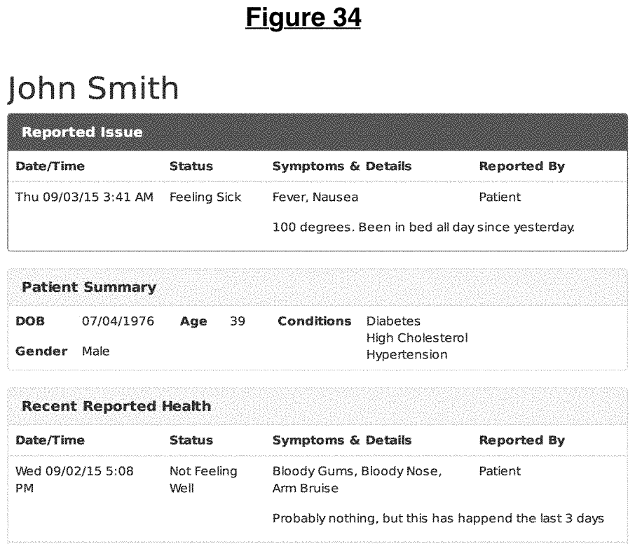

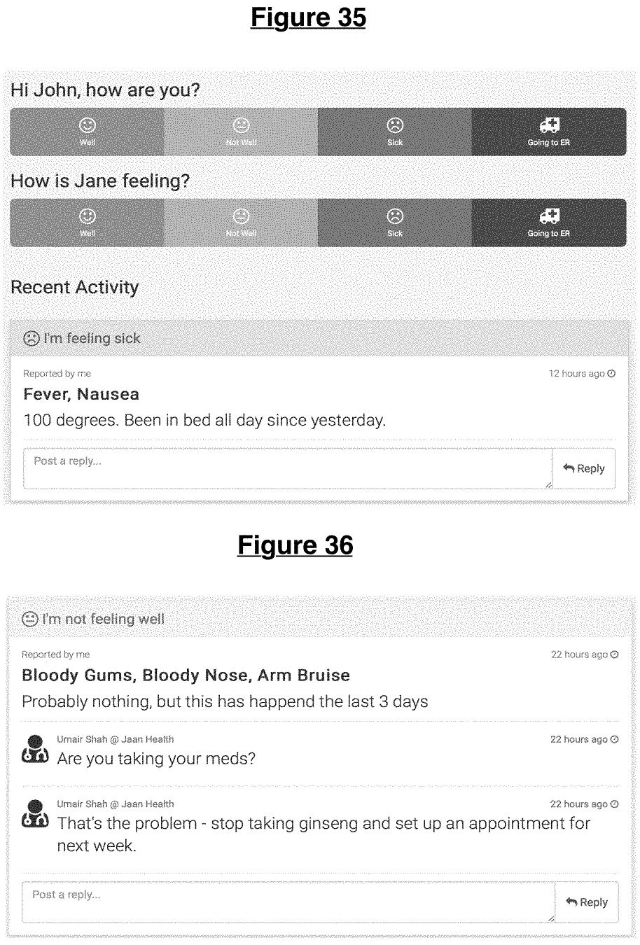

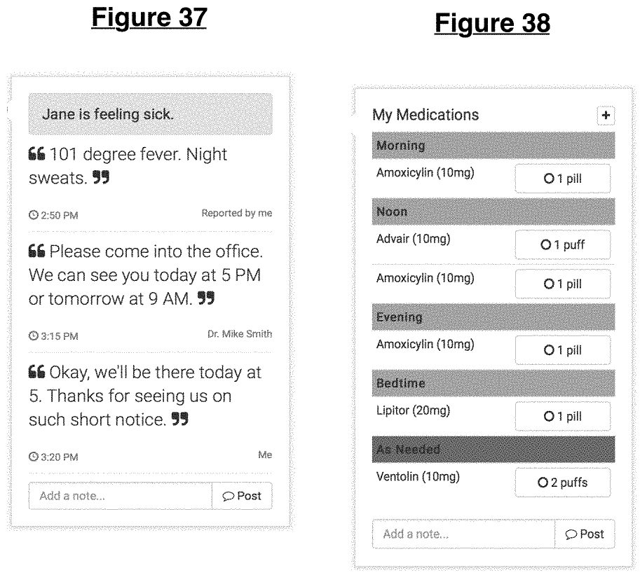

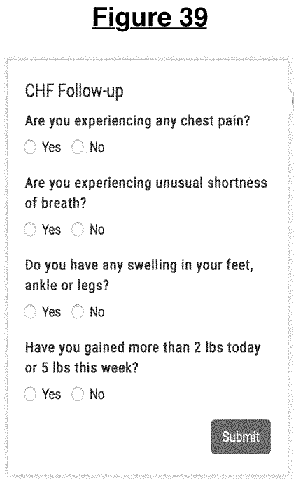

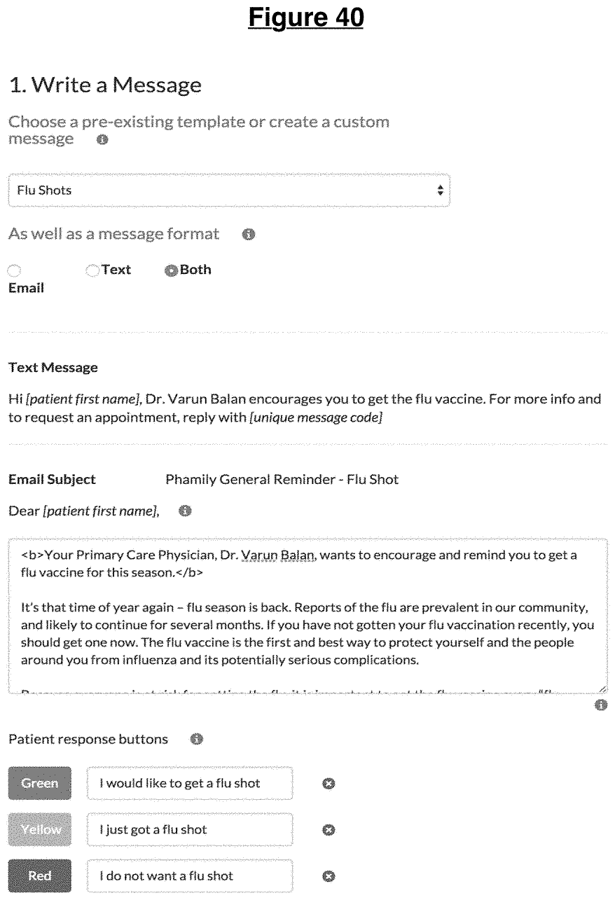

With reference to FIG. 2, there is shown an embodiment of a methodology 200 operable by a computing device for accessing a User Home Page. At step 202, a computing device may display or send instructions for displaying a Login/Signup Page. An example of a Login/Signup Page is shown in FIG. 16A. At step 204, a computing device receives the selection of Login or Signup. For example, a user upon seeing a Login/Signup Page, may then select Login or Signup. At step 206, if a computing device has received a selection of Login, the computing device may display or send instructions for displaying a Login Page. An example of a Login Page is shown in FIG. 16B. In various embodiments, a Login Page may request one or more of various forms of personal information, such as an email address, a unique user ID, an activation code, a pin code, biometric information, a password, etc. At step 208, if a computing device has received a selection of Signup, the computing device may proceed with a Signup process. For example, a Signup process may include performing one or more of the methodologies 300, 400, 500, or 600, described herein with respect to FIGS. 3-6. At the conclusion of the Signup Process, a computing device may next proceed to step 206 above or it may use information obtained during the Signup Process to proceed to step 210. At step 210, a computing device may receive and may store login information, such as from user entry, via the Signup Process, or by other sources. In some embodiments, the login information may be stored for automating the login process, thereby allowing for a computing device to avoid steps 202 to 210. At step 212, a computing device may display a User Home Page. An example of a User Home Page is shown in FIG. 16I.

With reference to FIG. 3, there is shown an embodiment of a methodology 300 operable by a computing device for obtaining User Registration information. At step 302, a computing device may display or send instructions for displaying a User Registration Page. An example of a User Registration Page is shown in FIG. 16C. In various embodiments, a User Registration Page may request one or more of various forms of personal information associated with a user, such as a first name, a last name, a date of birth, one or more email addresses (e.g., personal, work), one or more phone numbers (e.g., home, mobile), one or more street addresses (e.g., home, work), etc. In additional embodiments, a User Registration Page may also request a password and confirmation of the password. At step 304, a computing device may receive and may store the User Registration information as requested by a User Registration Page.

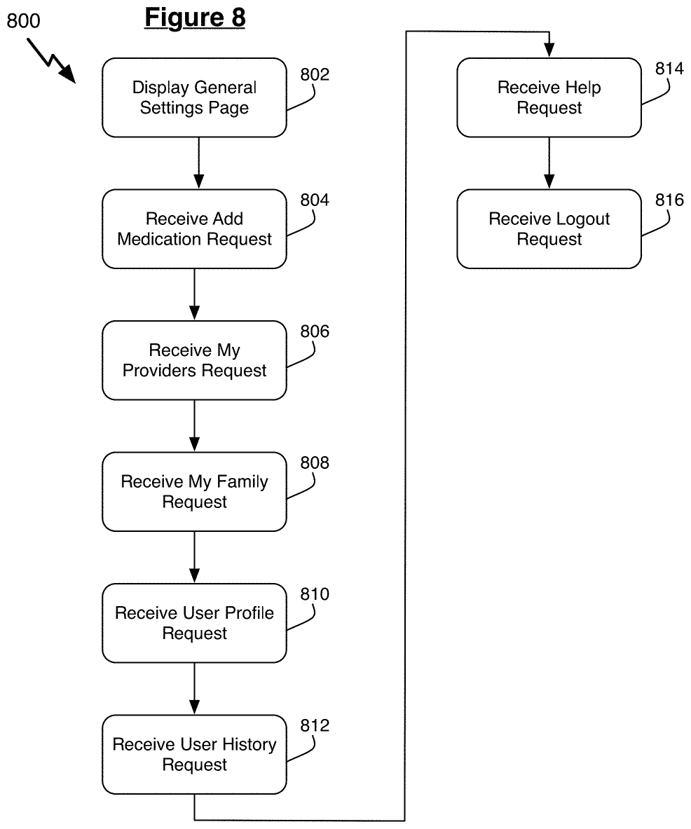

With reference to FIG. 4, there is shown an embodiment of a methodology 400 operable by a computing device for selecting or adding a Family Member. At step 402, a computing device may display or send instructions for displaying a My Family Page. An example of a My Family Page is shown in FIG. 16N. In various embodiments, a My Family Page may display a list of agents, such as family members, friends, or caretakers, associated with a user, which the user may then select. At step 404, a computing device may receive and may store the selection of an agent selected by a user, which may result in the agent being defined as an authorized agent in the user's record.

At step 406, a computing device may display or send instructions for displaying an Add Family Page. An example of an Add Family Page is shown in FIG. 16F. In various embodiments, an Add Family Page may request one or more of various forms of personal information of an agent, such as first name, last name, type of relationship, a date of birth, one or more email addresses (e.g., personal, work), one or more phone numbers (e.g., home, mobile), one or more street addresses (e.g., home, work), etc. At step 408, a computing device may receive and may store the Add Family Member information as requested by an Add Family Page. In some embodiments, if the information provided does not correspond to any user or agent, a message may be sent by a computing device inviting that person to become a user or agent using the contact information provided.

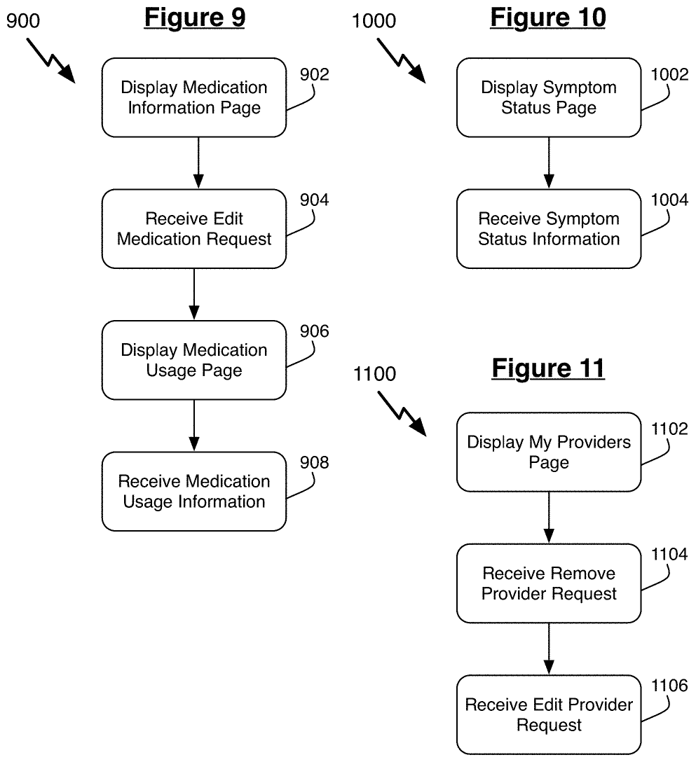

With respect to FIG. 5, there is shown an embodiment of a methodology 500 operable by a computing device for selecting or adding a provider (e.g., a physician, healthcare provider, case manager, or other healthcare services administrator). At step 502, a computing device may display or send instructions for displaying a Provider Selection Page. An example of a Provider Selection Page is shown in FIG. 16D. In various embodiments, a Provider Selection Page may display a list of providers, such as physicians, healthcare specialists, case managers, healthcare services administrators, etc., which the user may then select. At step 504, a computing device may receive and may store the selection of a provider associated with a user, which may result in the provider being defined as an authorized provider in the user's record.

At step 506, a computing device may display or send instructions for displaying an Add Provider Page. An example of an Add Provider Page is shown in FIG. 16E. In various embodiments, an Add Provider Page may request one or more of various forms of professional information associated with a provider, such as first name, last name, institution, type of practice, one or more email addresses, one or more phone numbers, one or more street addresses, one or more affiliations with hospitals, practice groups, or insurance, and so on. At step 508, a computing device may receive and may store the Add Provider information as requested by an Add Provider Page.

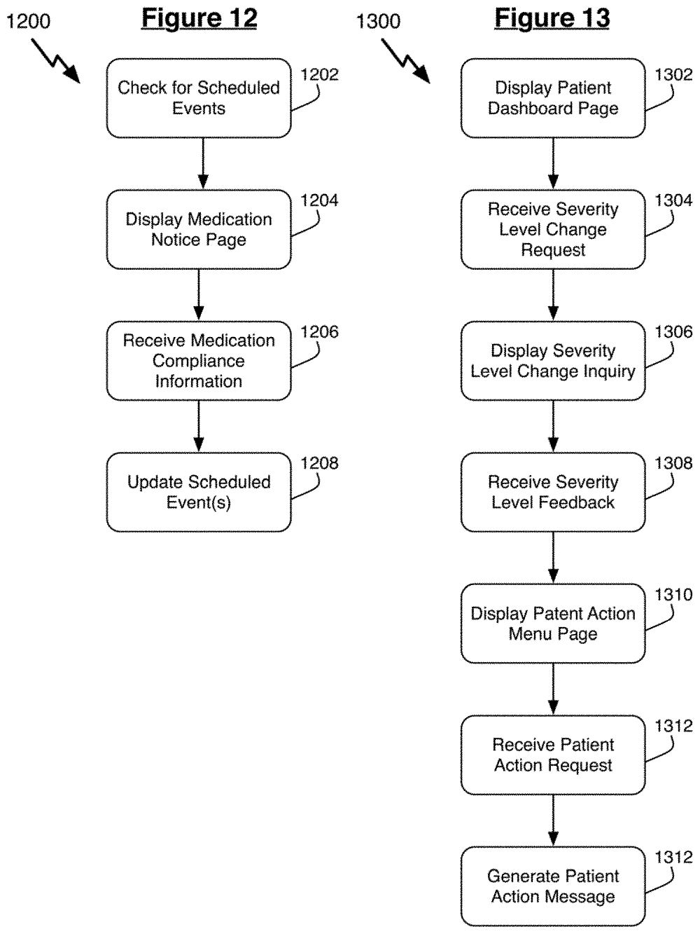

With respect to FIG. 6, there is shown an embodiment of a methodology 600 operable by a computing device for selecting or adding a medication (e.g., prescription drugs, over-the-counter medication, vitamins, supplements). At step 602, a computing device may display or send instructions for displaying a Medication Home Page. An example of a Medication Home Page is shown in FIG. 16G. In various embodiments, a Medication Home Page may show a list of medications for selection by a user. In additional embodiments, a Medication Home Page may allow a user to select a medication by scanning a barcode. In some embodiments, the listing of medications may use one or more of drug information, such as brand name, active pharmaceutical ingredient, national drug code or similar identifier, any other labeling information, manufacturer, etc. At step 604, a computing device may receive and may store a selection of a medication for a prescription, treatment, therapy, etc. At step 606, a computing device may display or send instructions for displaying a Medication Usage Page. An example of a Medication Usage Page is shown in FIG. 16H. In various embodiments, the Medication Usage Page may allow a user to select one or more parameters in association with a medication selected by the user, such as strength, dosage, frequency of use, quantity to be consumed, start date, number of days, end date, scheduled time for administration of the medication, setting of reminder notices, the location where the drug was obtained, etc. In some embodiments, the Medication Usage Page may suggest default parameters, such as scheduled time(s) for administration of a medication, based on information associated with a medication contained in its medication record. For example, a medication record may indicate that a medicine is best consumed in the morning, a certain time before sleep, that minimum time intervals be maintained between application, etc. In some embodiments, a user may able to adjust these default preferences in accordance with the user's preferred schedule (e.g., changing user's wake up time from 8 AM to 4 AM). At step 608, a computing device may receive and may store the Medication Usage information as requested by a Medication Usage Page. In some embodiments, the setting of reminder notices may further include specifying agents to receive or not receive such notices.

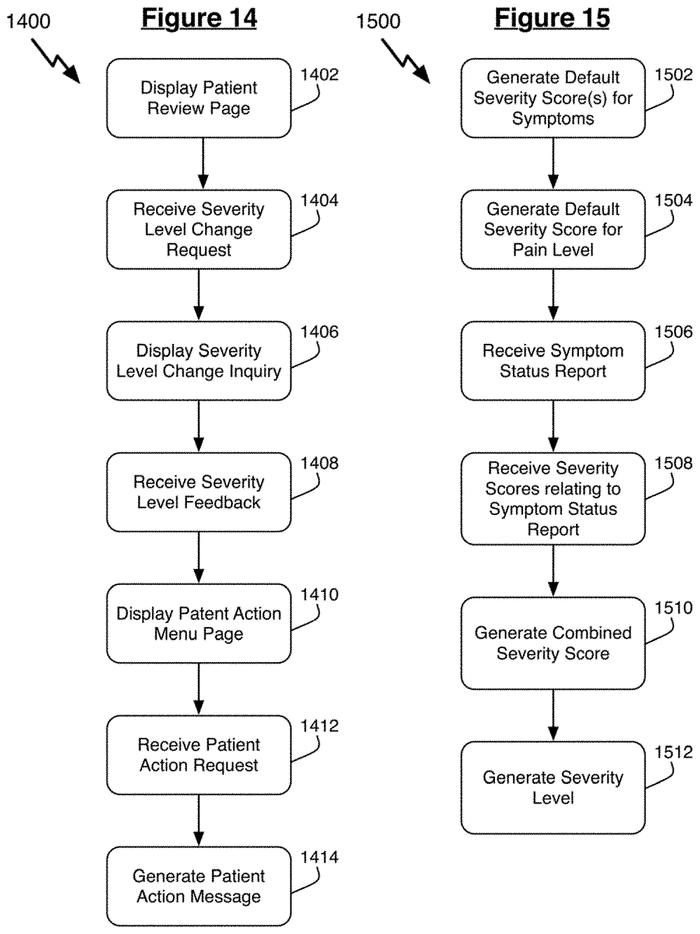

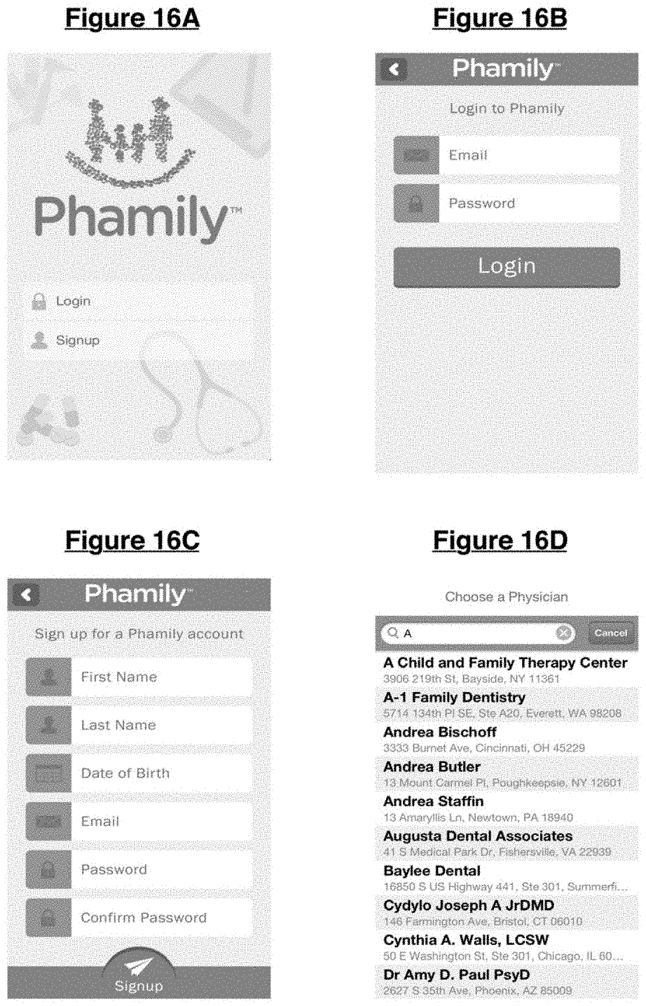

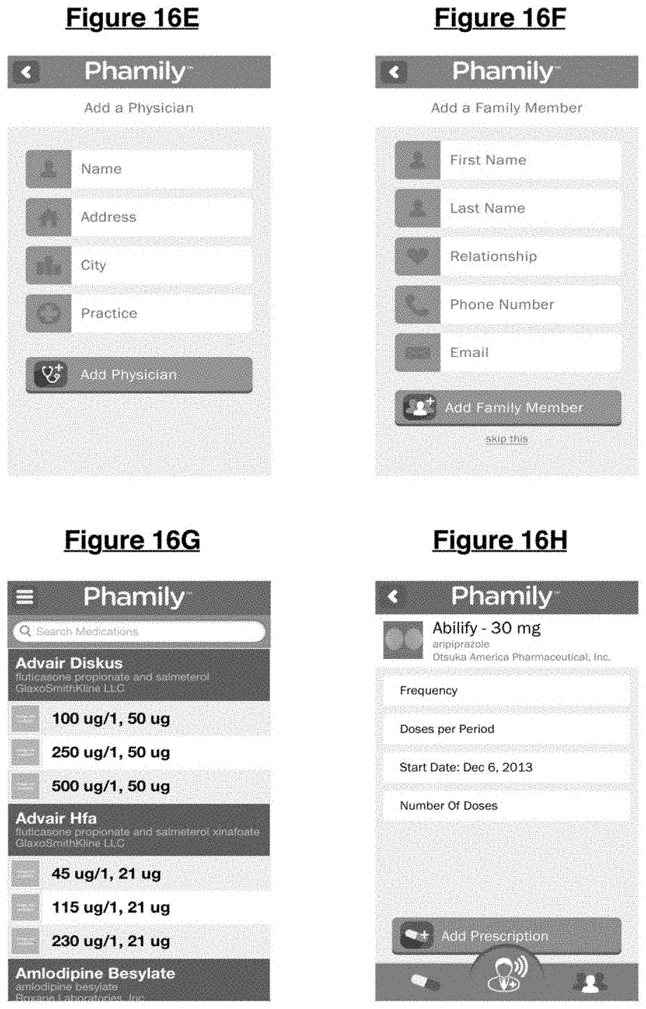

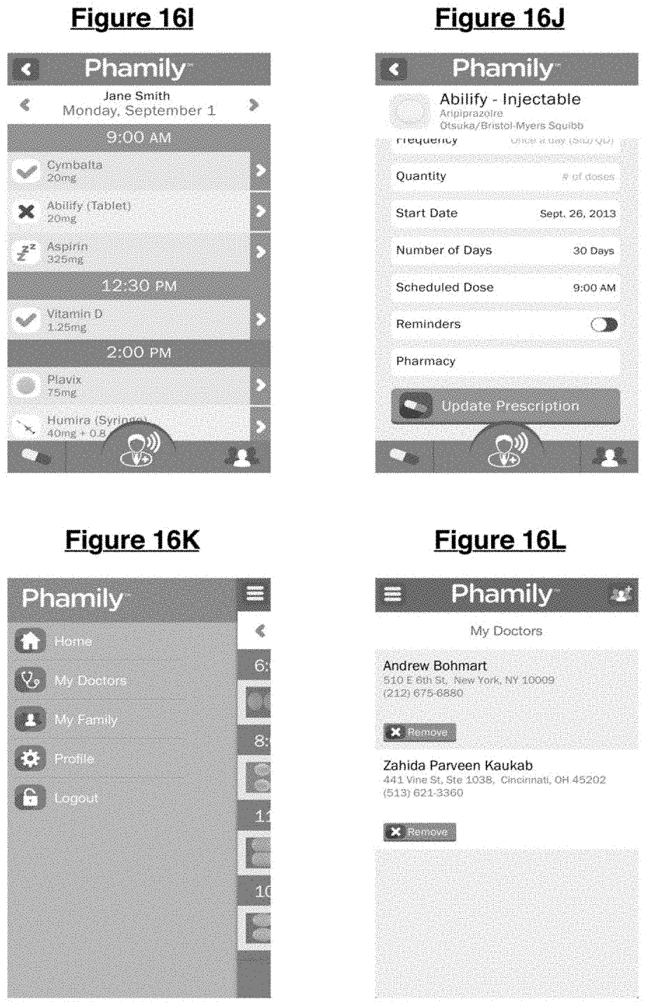

With respect to FIG. 7, there is shown an embodiment of a methodology 700 operable by a computing device for accessing or editing information from a User Home Page. At step 702, a computing device may display or send instructions for displaying a User Home Page. An example of a Patient Home Page is shown in FIG. 16I. In various embodiments, a User Home Page may show one or more of a user's name, a schedule of medications to be taken by the user, a menu item that allows the user to access General Settings (described further below), a menu item for each medication scheduled that allows the user to edit or review Medication Usage information for each such medication scheduled, a menu item for adding additional medications to the user's account, a menu item for providing symptom status, or a menu item for accessing a My Family Page. At step 704, a computing device may receive and may store a request for accessing the General Settings, which may lead the computing device to perform the steps of methodology 800 described herein with respect to FIG. 8. At step 706, a computing device may receive and may store a request for adding a medication, which may lead the computing device to perform the steps of methodology 900 described herein with respect to FIG. 9. At step 708, a computing device may receive and may store a request to review and edit Medication Usage Information, which may lead the computing device to perform the steps of methodology 900 as described herein with respect to FIG. 9. At step 710, a computing device may receive and may store a request to access a My Family page, which may lead the computing device to perform the steps of methodology 400 described herein with respect to FIG. 4. At step 712, a computing device may receive and may store a request to provide a Symptom Status report, which may lead the computing device to perform the steps of methodology 1000 as described herein with respect to FIG. 10. In some embodiments, one or more of the steps described above may be accessible from other Pages described herein.

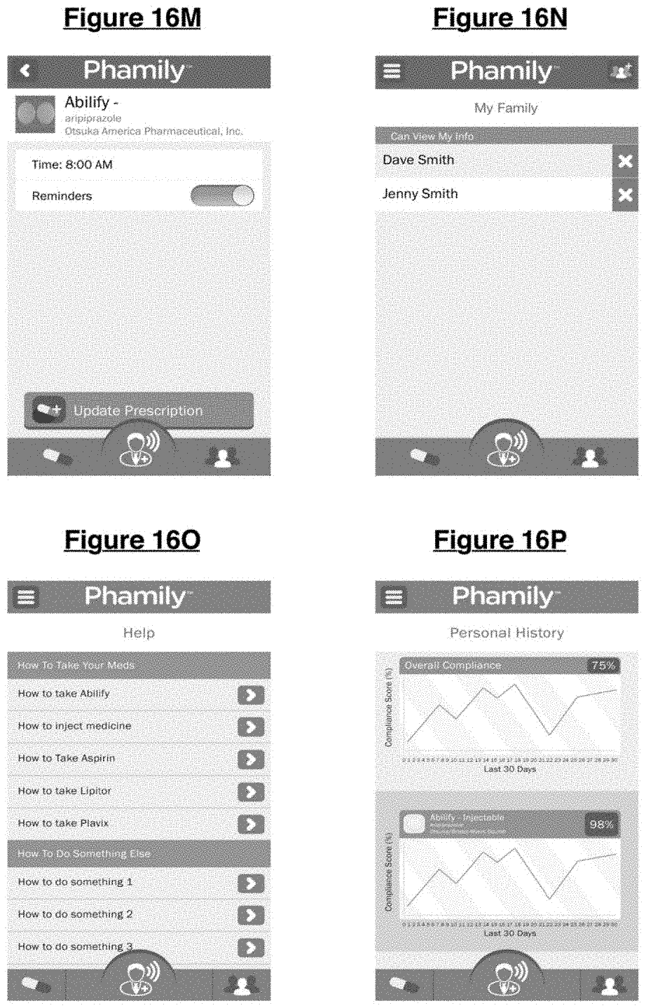

With respect to FIG. 8, there is shown an embodiment of a methodology 800 operable by a computing device for accessing or editing information from a General Settings Page. At step 802, a computing device may display or send instructions for displaying a General Settings Page. An example of a General Settings Page is shown in FIG. 16K. In various embodiments, a General Settings Page may show one or more of a menu item for adding additional medications to a user's account, a menu item for accessing a My Family Page, a menu item for accessing a User Profile Page, a menu item for accessing a User History Page, a menu item for accessing a Help Page, a menu item for requesting a logout, etc. At step 804, a computing device may receive and may store a request for adding a medication, which may lead the computing device to perform the steps of methodology 600 described herein with respect to FIG. 6. At step 806, a computing device may receive and may store a request to access a My Providers page, which may lead the computing device to perform the steps of methodology 1100 described herein with respect to FIG. 11. At step 808, a computing device may receive and may store a request to access a My Family page, which may lead the computing device to perform the steps of methodology 400 described herein with respect to FIG. 4. At step 810, a computing device may receive and may store a request to access a User Profile Page, which may lead the computing device to perform one or more steps of methodology 300 described herein with respect to FIG. 3.

With respect to FIG. 9, there is shown an embodiment of a methodology 900 operable by a computing device for accessing medication information or editing medication usage information. At step 902, a computing device may display or send instructions for displaying a Medication Information Page. An example of a Medication Information Page is shown in FIG. 16M. In various embodiments, a Medication Information Page may show one or more forms of information regarding a medication associated with a user, such as brand name, active pharmaceutical ingredient, manufacturer, strength, scheduled time of dosage, last actual time of dosage, strength of medication, amount of dosage, frequency of medication, medical instructions, cautions, other labeling information, a graphical display of the user's adherence to medication over a period of time (e.g., one week, one month), etc. In additional embodiments, a Medication Information Page may also show a menu selection for editing the Medication Usage information associated with the medication shown. At step 904, a computing device may receive and may store an Edit Medication request, which may lead the computing device to perform step 906. At step 906, a computing device may display or send instructions for displaying a Medication Usage Page using previously stored information regarding the selected medication. An example of a Medication Usage Page is shown in FIG. 16H. In various embodiments, the Medication Usage Page may allow a user to select or edit one or more parameters in association with a medication selected by the user, such as dosage, frequency of use, quantity to be consumed, start date, number of days, end date, scheduled time for administration of the medication, setting of reminder notices, the location where the drug was obtained, etc. At step 908, a computing device may receive and may store the Medication Usage information as requested by a Medication Usage Page.

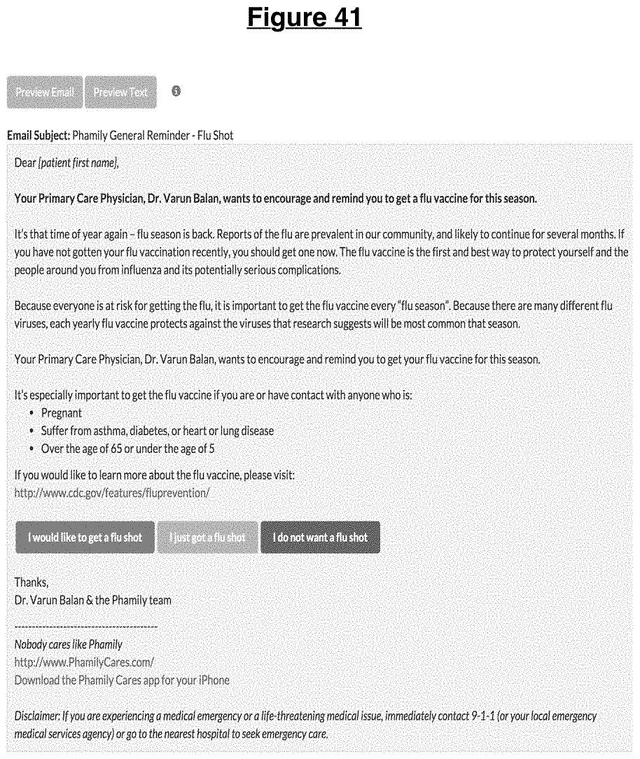

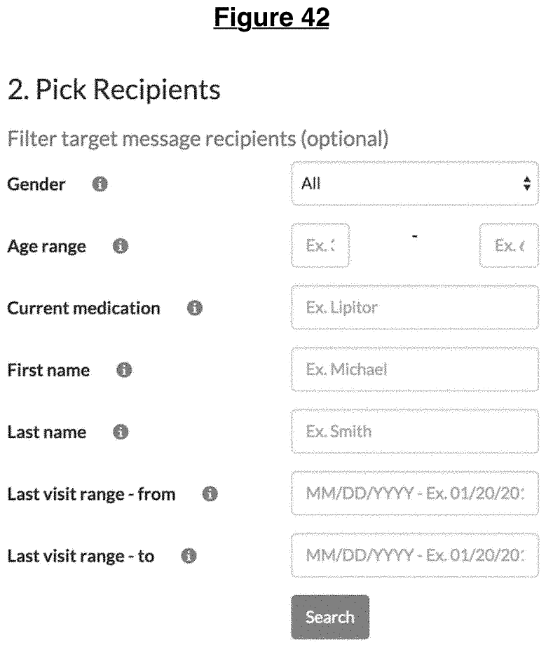

With respect to FIG. 10, there is shown an embodiment of a methodology 1000 operable by a computing device for accessing or editing symptom status. At step 1002, a computing device may display or send instructions for displaying a Symptom Status Page. Examples of a Status Symptom Page are shown in FIGS. 17A through 17F and 18A through 18B. In various embodiments, the Symptom Status Page may allow a user to select one or more symptoms describing their current or recent physical or mental condition(s), such as nausea, fever, coughing, fatigue, etc. In additional embodiments, the Symptom Status Page may also allow a user to add additional symptoms other than those shown on the Symptom Status Page or allow the user to use a sliding scale or numeric value within a range to indicate a general sense of well-being (e.g., 2 is “It hurts”, 5 is “Can't tolerate it”) or may show graphical representations of well-being, such as color-coded smiley faces that range from showing happiness to an extreme state of pain. In some embodiments, symptoms selected for the Symptom Status Page may be based on the likelihood of known side effects associated with a medication or the likelihood of recorded side effects by users of a medication. For example, the Symptom Status Page may use information regarding the likelihood of side effects associated with a medication using Medication Records stored in Database Component 122 of System 120.

At step 1004, a computing device may receive and may store the Symptom Status information as requested by a Symptom Status Page. In some embodiments, a computing device may perform an additional step upon receiving Symptom Status information of sending part or all of such information as a notification to authorized providers or agents, such as those listed on a user's My Provider Page or My Family Page, to receive such notices.

With respect to FIG. 11, there is shown an embodiment of a methodology 1100 operable by a computing device for accessing or editing providers. At step 1102, a computing device may display or send instructions for displaying a My Providers Page. An example of a My Providers Page is shown in FIG. 16L In various embodiments, the My Providers Page may show information relating to a provider, such as a provider's first name, last name, institution, type of practice, phone number, email address, street address, etc. In additional embodiments the My Providers Page may allow the user to remove the provider from the My Providers Page or allow the user to edit the information of a provider listed on the My Providers Page. At step 1104, a computing device may receive and may store a request to remove the provider from the user's My Provider Page, which may lead the computing device to remove the status of provider in relation to that user as an authorized provider. At step 1106, a computing device may receive and may store a request to edit the information of a provider associated with a user's account, which may lead the computing device to perform the steps 506 and 508 of methodology 500 described herein with respect to FIG. 5, except that the Add Provider Page may be pre-populated with information regarding the provider already stored in the user's account.

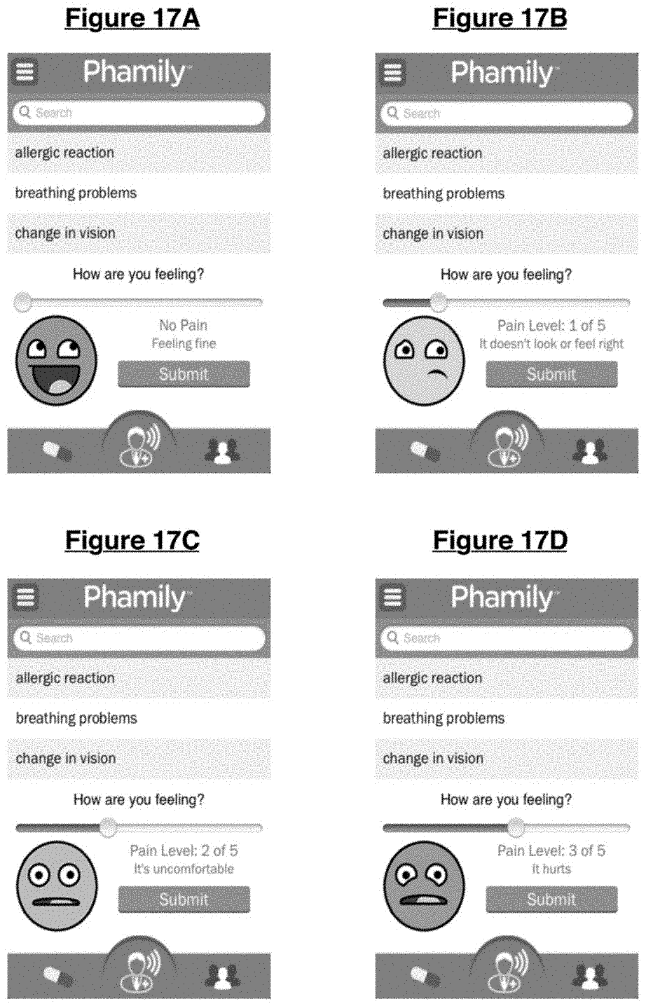

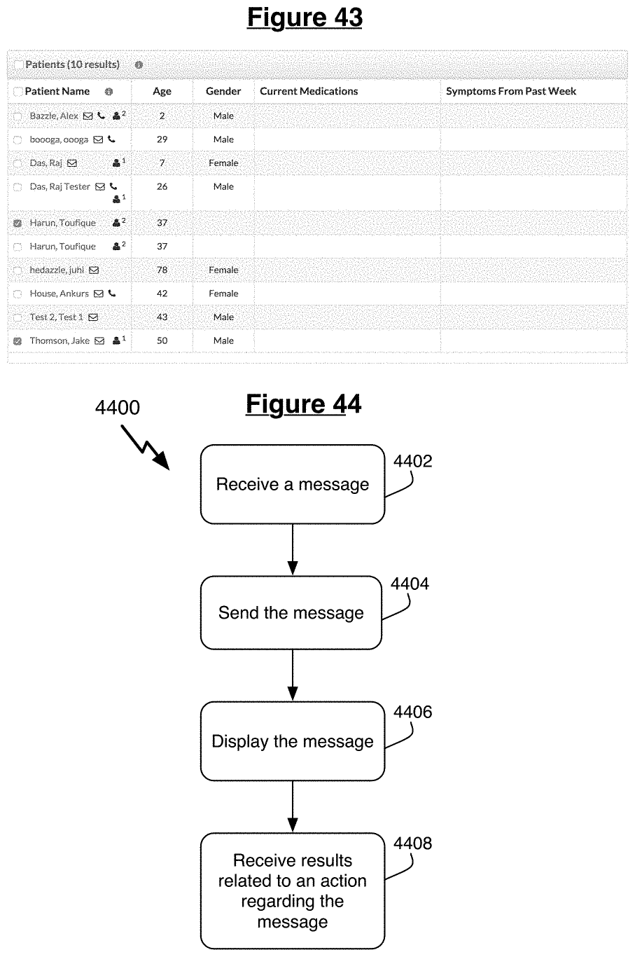

With respect to FIG. 12, there is shown an embodiment of a methodology 1200 operable by a computing device for notifying a user of a need to take a medication. At step 1202, a computing device may check whether any medications are scheduled for notice. At step 1204, if a computing device finds that one or more medications are scheduled for notice, it may display or send instructions for displaying a Medication Notice Page. Examples of a Medication Notice Page are shown in FIGS. 19A and 19B. In various embodiments, a Medication Notice Page may show one or more forms of information relating to a medication, such as the name of the medication(s) that should be taken, instructions for taking the medication(s), the dosage(s) of the medication(s) to be taken, cautions, the time scheduled for taking the medication(s), etc. In additional embodiments, the Medication Notice Page may further allow a user to select a menu item indicating that a medication was taken, that the time for taking a medication should be delayed, that a medication was not taken, etc. At step 1206, a computing device may receive and may store Medication Compliance Information, such as whether the user selected that a medication was taken, that the time for taking a medication was delayed, that a medication was not taken, etc. At step 1208, if the user selected to delay the medication, a computing device may update when a medication is scheduled for notice.

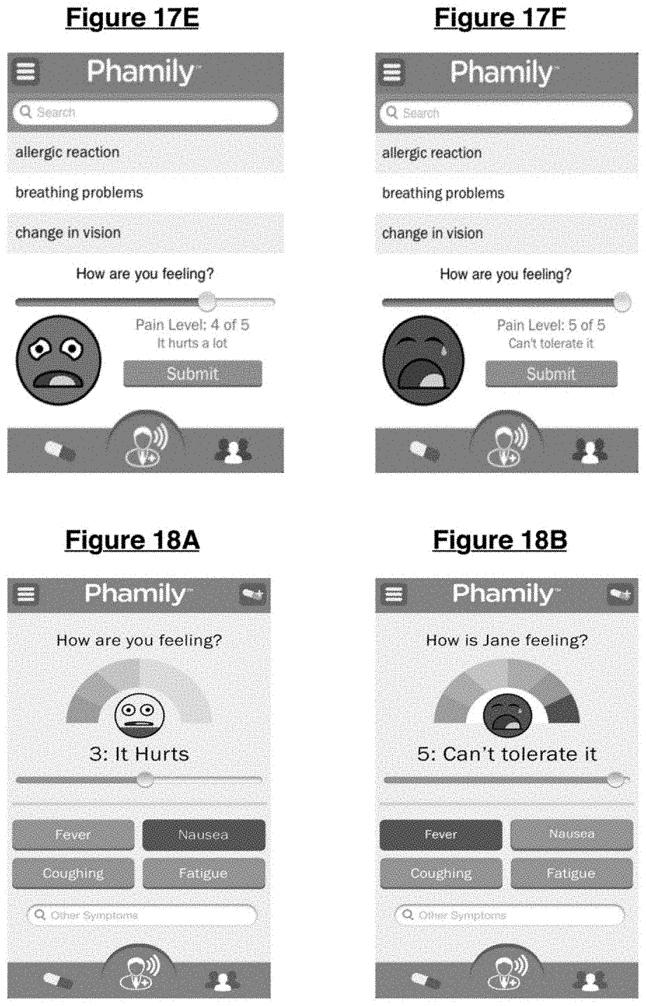

With respect to FIG. 13, there is shown an embodiment of a methodology 1300 operable by a computing device for providing an overview of user status to a provider. At step 1302, a computing device may display or send instructions for displaying a Patient Dashboard Page. An example of a Patient Dashboard Page is shown in FIG. 21. In various embodiments, a Patient Dashboard Page may show one or more forms of information for each user associated with an authorized provider, such as the name of each user, the condition of each user, one or more symptom statuses of each user, one or more severity levels corresponding to each user, the pain level associated with the symptom, who reported a user's symptoms, the time of when each symptom status was recorded, etc. In additional embodiments, the Patient Dashboard Page may allow the provider to sort the information presented regarding users according to specific criteria, such as the nature of severity level (e.g., show only severe, moderate, or new symptoms), particular symptoms reported, or the extent of satisfactory or unsatisfactory adherence to a prescription, treatment, therapy, etc. by users (e.g., show only users who fail to take prescribed medications each day, show users experiencing significant declines in adherence, users who have run out of medication, etc.). At step 1304, a computing device may allow a provider to select a reported security level and indicate a different preferred security level. At step 1306, a computing device may allow a provider to provide information regarding why the preferred security level should be altered (e.g., symptom is high risk and not low risk). At step 1308, a computing device may receive and may store the preferred security level and also any information relating to why it was changed by the provider.

At step 1310, a computing device may display or send instructions for displaying a Patient Action Menu Page so as to allow a provider to select an action that may occur with respect to a user, such as requesting that a call be scheduled between the user and the provider, that an office visit be scheduled between the user and the provider, that an urgent request be sent to the user on behalf of the provider (e.g., go to the emergency room, cease taking all or certain medications), that a user continue to monitor his or her situation and report any changes for the worse to the provider, etc. At step 1310, a computing device may receive a Patient Action Request as described above. At step 1312, a computing device may forward the request or generate a Patient Action Message using an appropriate method to contact a user or his or her authorized agent(s) or provider(s). For example. Notification/Messaging Component 130 may generate a Patient Action Message in a variety of forms, such as push notifications, emails, text messages, or robo-calls containing instructions or requests relevant to the selected action for a user by a provider. In some embodiments, such a Patient Action Message may be sent not only to a user by a computing device, but also to his or her authorized agent(s) or authorized provider(s) as well.

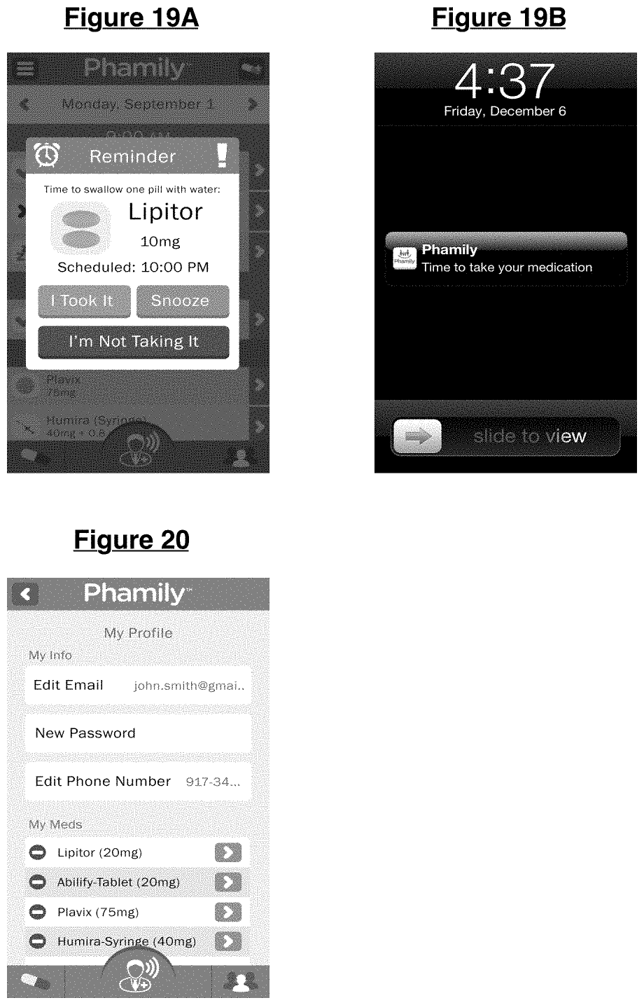

With respect to FIG. 14, there is shown an embodiment of a methodology 1400 operable by a computing device for providing an overview of particular user information to a provider. At step 1402, a computing device may display or send instructions for displaying a Patient Review Page. An example of elements that constitute a Patient Review Page is shown in FIGS. 22A through 22E. In various embodiments, a Patient Review Page may show one or more forms of information for a user associated with an authorized provider, such as the user's name, user's address, user's age, user's insurance, user's phone number, user's nearest or preferred pharmacy, information about the user's procedure or visit history, information about the user's upcoming procedures or appointments, etc. In additional embodiments, the Patient Review Page may also show current or past information regarding medications for a user, such as brand name, dosage, strength, date of prescription/treatment/therapy/etc., duration of prescription/treatment/therapy/etc., prescribing provider, number of refills, user's rate of adherence, etc. In additional embodiments, the Patient Review Page may also show scalable charts or graphs indicating the adherence of the user to certain medications over time or the number of side effects, symptoms, or severity levels experienced by the user over a period of time. Steps 1404 through 1412 may occur in methodology 1500 in a similar manner as described above with respect to steps 1304 through 1312 of methodology 1300.

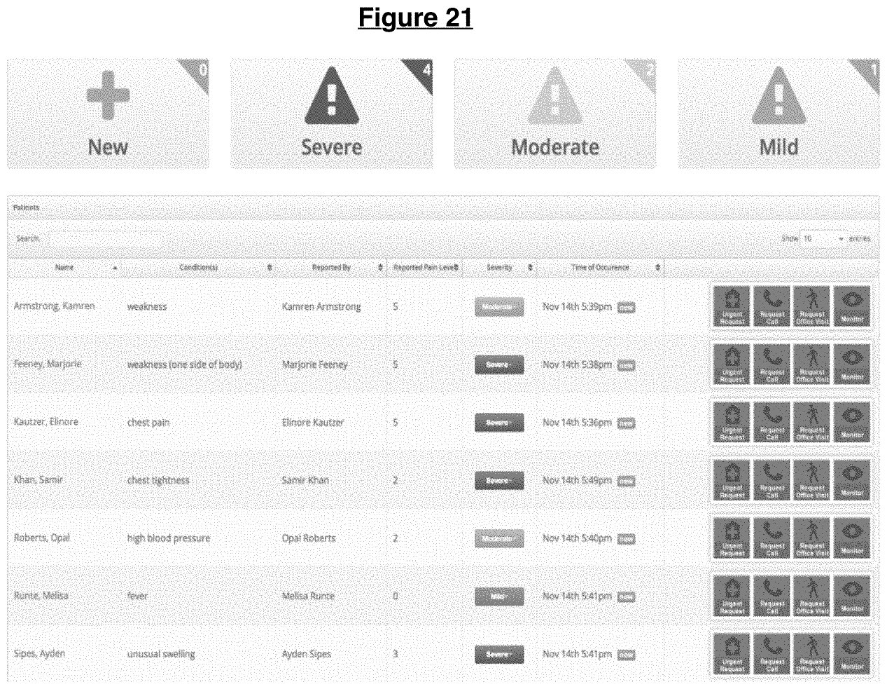

With respect to FIG. 15, there is shown an embodiment of a methodology 1500 operable by a computing device for calculating severity levels and scores in relation to medication or Symptom Status information. At step 1502, a computing device may generate and may store default severity scores for the symptoms that may be reported in a Symptom Status Report. For example, a symptom such as chest pain may be generally scored with a high value (e.g., 5), while itchiness may be generally scored with a low value (e.g., 1). In some embodiments, a computing device may further adjust the default severity scored based on information associated with a medication (e.g., certain symptoms normally considered low risk may indicate high risk when using certain medications) or by analyzing feedback from providers regarding the appropriate value for a severity score. At step 1504, a computing device may generate and may store default severity scores for the level of pain that may be reported in a Symptom Status Report. For example, a severity score may correspond with the level of pain selected by a user (e.g., a pain level of 2, corresponding to “unwell”, may have a severity score of 2). In some embodiments, a computing device may further adjust the default severity scored based on information associated with a user (e.g., the user may underemphasize or overemphasize his or her level of pain) or by analyzing feedback from providers regarding the appropriate value for a severity score. At step 1506, a computing device may receive a Symptom Status Report. At step 1508, a computing device may request or retrieve severity scores for the symptoms or pain levels reports or both based on a Symptom Status Report. In some embodiments, a computing device may receive adjusted severity scores or adjust the severity scores as described herein based on medications being taken by a user, analysis of feedback from providers, etc. At step 1510, a computing device may generate and may store a combined severity scored based on severity scores received or adjusted, such as severity scores based on a pain level of a user or reported symptoms or both. In some embodiments, the combined severity score may be calculated by adding or averaging the individual severity scores (e.g., pain level severity score and symptom severity score(s)). In other embodiments, more advanced statistical methods may be used to create combined severity scores based on two or more severity scores or to also include analysis based on previously generated severity levels. At step 1512, a computing device may generate and may store, such as in a User Record, a severity level based on the value of a combined severity score. For example, if a combined severity score is within certain ranges of values, it may be designated as mild (e.g., combined severity score within 0 to 2), moderate (e.g., combined severity score above 2 to below 4), or serious (e.g., combined severity score at or above 4). In some embodiments, the criteria for generating severity levels may be adjusted by analyzing feedback from providers.

With respect to FIG. 23, there is shown an embodiment of a methodology 2300 operable by a computing device for managing pharmacy/patient interactions. At step 2302, a computing device may receive information relating to a user's medication risks (e.g., allergies) and may store the information, such as in a User Record. At step 2304, a computing device may display or send instructions for displaying a Prescription Entry Page. An example of elements that constitute a Prescription Entry Page is shown in FIG. 25A. In various embodiments, a Prescription Entry Page may show one or more information entry fields for a patient's prescription, such as the medication's generic name, brand name, or active ingredient; the strength of the medication; the medication's labeler or brand; the form of dosage to be used with the medication; the quantity of medication to be provided; the frequency that the medication should be used; any special instructions relating to the medication; when to take the medication (e.g., after breakfast); the reason for the medication; the person who prescribed the prescription; and any other information relating to the medication. At step 2306, a computing device may receive and may store a request for adding a medication, such as in a User Record. At step 2308, a computing device may display or send instructions for displaying a Patient Medication List Page. An example of elements that constitute a Patient Medication List Page is shown in FIG. 25B. As shown in FIG. 258, a computing device may use information received in steps 2302-2306 to create the Patient Medication List Page, or retrieve such information from a User Record or from other sources. In some embodiments, a computing device may also access a database containing images, labels, or other visual representations to aid a pharmacist in distinguishing between medications and display or send instructions for displaying such images, labels, or other visual representations. At step 2308, a computing device may display or send instructions for displaying a Patient Medication Report. An example of elements that constitute a Patient Medication Report is shown in FIG. 27. As shown in FIG. 27, a computing device may use information received in steps 2302-2306 to create the Patient Medication Report, or retrieve such information from a User Record or from other sources.

With respect to FIG. 24, there is shown an embodiment of a methodology 2400 operable by a computing device for managing a patient action plan. At step 2402, a computing device may display or send instructions for displaying an Action Plan Entry Page. An example of elements that constitute an Action Plan Entry Page is shown in FIG. 26A. In various embodiments, a Prescription Entry Page may show one or more information entry fields for a patient's action plan, such as a topic, a description of an issue, and a proposed resolution of the issue. At step 2404, a computing device may receive and may store a request for adding an action plan, such as in a User Record. At step 2406, a computing device may display or send instructions for displaying an Action Plan List Page. An example of elements that constitute an Action Plan List Page is shown in FIG. 26B. As shown in FIG. 26B, a computing device may use information received in steps 2402-2404 to create the Action Plan List Page, or retrieve such information from a User Record or from other sources. At step 2408, a computing device may display or send instructions for displaying an Action Plan Report. An example of elements that constitute an Action Plan Report is shown in FIG. 28. As shown in FIG. 28, a computing device may use information received in steps 2402-2404 to create the Action Plan Report, or retrieve such information from a User Record or from other sources. In some embodiments, the Action Plan Report may take the form shown in FIG. 29, thereby allowing for a pharmacist to receive approval of an action plan from a physician.

In some embodiments, the Action Plan Entry Page may allow for the entry of a questionnaire (e.g., a checklist of required activities). In such embodiments, the system and methods described herein may present the questionnaire to a user and may receive answers from the user in response to the questionnaire. Alternatively, the system and methods described herein may record a lack of response to the questionnaire. In further embodiments, the entry of a questionnaire may also allow for the entry of instructions relating to when the questionnaire should be presented to the user (e.g., send every day, send in two weeks, etc.). In certain aspects, the answers to the questionnaire may trigger an automated response by the systems and methods disclosed herein (e.g., call your doctor, go to hospital immediately, etc.).

In additional embodiments, Notification/Messaging Component 130 may provide further messaging functionality. For example, Notification/Messaging Component 130 may retain a history of messages. In some embodiments, Notification/Messaging Component 130 may store messages in Database Component 122. In some embodiments. Notification/Messaging Component 130 may provide further functionality allowing for the display, processing, creation, and editing of messages. In some embodiments, based on the recipient of the message, Notification/Messaging Component 130 may retrieve a record from Database Component 122 indicating who is to receive the message (e.g., patient, patient and patient's agent(s), provider) and may also determine for the message sent the allowed method for sending of the message (e.g., patient may specify email only, patient's agent may specify text message only). Depending on the allowed method of sending the message, Notification/Messaging Component 130 may format the message in a manner for a suitable for method of sending. For example, if the message requires a yes or no response from the recipient, an email message may use response buttons as described below, while a text message may use numeric responses (e.g., “Reply 1 for Yes, 0 for No.”). If the message, however, requires a more complex response, such as rescheduling an appointment to a particular time, an email or text message may use customizable alphanumeric responses.

With respect to FIG. 30, there is shown another example of a Provider Dashboard Page. A computing device may retrieve a collection of messages, such as those belonging to a particular provider, from an internal cache on Notification/Messaging Component 130 or from Database Component 122 and may generate a set of message summaries based on each message. Further, a computing device may provide a set of actions (which may be stored or determined by Notification/Messaging Component 130) that can be performed with respect to each message. As shown in FIG. 30, such actions may include replying to a message, archiving a message, downloading a message, printing a message, or requesting a consultation (e.g., an appointment with the provider) to be scheduled. A computing device may also receive status indicators relative to the content or type of the message, such as by consulting Notification/Messaging Component 130. For example, Notification/Messaging Component 130 many contain instructions that a message summary be shown in blue for low-priority messages (e.g., emails not requiring a medical review), green for appointments, and red for high-priority messages (e.g., emails requiring a medical review). A computing device may then send a set of message summaries, a set of actions associated with each message, and a set of status indicators relative to each message for display, such as by Notification/Messaging Component 130. A computing device may then display the set of message summaries, the set of actions associated with each message, and the set of status indicators relative to each message as shown in FIG. 30. An action associated with a message may then be received by a computing device and may then be sent via Notification/Messaging Component 130.

With respect to FIG. 31, an example of a Provider Message Options Page is shown. The Provider Message Options Page may be shown for example when a provider has chosen to reply to a message. A computing device based on the message may include a message summary, status indicators or actions as described above with respect to the message. As shown in FIG. 31, a message summary may include a patient's name, the patient's symptoms, a date and time when the patient's message was received, and further details provided by the patient in the patient's message. A computing device may also include actions relating to typical responses to a patient condition. For example, such actions may include selecting options including monitor, schedule, call, come in, urgent, or other options, which may be implemented as follows: if “Monitor” is selected, a computing device may provide a standard response instructing the patient to continue monitoring his or her condition or provide further updates if the condition becomes more severe; if “Schedule” is selected, a computing device may provide a standard response instructing the patient to schedule an appointment; if “Call” is selected, a computing device may provide a standard response instructing the patient to call the provider; if “Come in” is selected, a computing device may provide a standard response instructing the patient to come to a provider's location; if “Urgent” is selected, a computing device may provide a standard response instructing the patient to call 911 or go to an emergency room.

A computing device may automatically enter such a standard response as text into the area shown in FIG. 31 designated for text entry (i.e., “Send a message . . . ” Further, the standard response may include field codes to automatically insert information regarding the patient, symptoms, provider, etc. For example, a standard response generated after selecting “Come in” may be “(Patient_first_name), please come into our office located at (Provider_Address_1).” Upon sending the message (e.g., by hit hitting reply), a computing device may substitute the relevant information for any field codes. In addition, a computing device may provide a preview function that shows how a response using field codes may appear prior to sending a message. Further, a computing device may allow providers to define custom actions and associated standard responses not shown in FIG. 31. For example, a provider may wish to include options such as “R-Brown”, which may be shown under the “More” action in FIG. 31, to refer patients to contact a different provider (e.g., Dr. Brown).

With respect to FIG. 32, an example of a Provider Message Draft Page is shown. A computing device may provide the ability to send a message to a patient or a patient's agent without having to reply to a prior message. One or more patients may be searched for and selected via the patient field shown in FIG. 32. For example, a provider could type in the name of a patient's last name, select the patient by full name, and a computing device may accordingly fill in the patient field shown in FIG. 32. In addition, a computing device may allow providers to define a group name for a set of patients, which may be either static or dynamic. For example, a provider may define a group name “Pre-transfer” for a specific group of patients that transferred with the provider to a new clinic. As another example, a provider may define a group name “Diabetes40” for a specific group of patients that have Diabetes and are over the age of 40. In the case of a dynamic group name, a computing device may access Database Component 122 to obtain patients matching the criteria specified by a group name when a provider chooses to send the message and then send a message to such patients. As described above with FIG. 31, messages drafted by providers may also utilize field codes.

With respect to FIG. 33, an example of a Provider Appointment Reminder Page is shown. As shown in FIG. 33, the Provider Appointment Reminder Page may utilize the same approaches as described above to select a patient or a group name and to enter messages. FIG. 33 also shows the ability to enter a date/time for an appointment. A provider may specify a date/time for an appointment, which may then be substituted by a computing device based on a date/time field code in the message. In some embodiments, a computing device may automatically retrieve a patient's next appointment from Database 122 or an external system for managing patient appointments. In further embodiments, a computing device may allow a provider to select from an available list of available appointments, such as by selecting the clock symbol.

With respect to FIG. 34, an example of a Patient Interaction Report is shown. A computing device may allow a provider to select a set of messages to create a report relating to the treatment of a patient. A computing device based on the one or more selected messages may present a summary of reported issues. For example, if a patient sent a set of messages relating to having the flu, the provider may select that set of messages to automatically generate a summary of reported issues provided by the patient (e.g., fever, nausea, coughing). As shown in FIG. 34, an example of the information automatically generated in a summary of reported issues may include the date/time of a message, a message status, symptoms and details reported in the message, and who sent the message (e.g., the patient or the patient's agent). In addition, a Patient Interaction Report may allow a provider to generate a report relating to the treatment of a patient that correlates with established medical billing codes to generate a billing report.

A computing device may also automatically generate a patient summary for the report relating to the treatment of a patient. As shown in FIG. 34, an example of the information automatically generated in a patient summary may include a patient's date of birth, gender, age, and conditions. In addition, a patient summary may include billing information, referral information, insurance information, etc. A computing device may also automatically determine any messages that relate to the messages selected by the provider (e.g., all replies that are related to selected messages) to automatically generate a summary of recent reported health. As shown in FIG. 34, the summary of recent reported health may be generated on such related messages in a manner similar to the summary of reported issues. A computing device may also automatically include in the Patient Interaction Report any disclaimers, instructions, or other information required by another entity, such as those that may be required by government regulation (e.g., a statement stating that the record must be retained for 2 years), or that may be useful in dealing with patient record management (e.g., a unique identifier corresponding to the Patient Interaction Report). A computing device may automatically retrieve any information required to generate a Patient Interaction Report from Database 122 or from external systems maintained by a provider. A computing device may also allow providers to edit a template for generating a Patient Interaction Report, such as by allowing providers to modify the summaries described above. For example, a provider may edit the template to list any agents of the patient and their contact information in the patient summary by using field codes.

With respect to FIG. 35, an example of a Patient Dashboard Page is shown. As shown in FIG. 35, a computing device may receive and store an indication of how a patient or patients are feeling. In addition, a computing device may also receive and store a message entered by a patient. Further, if a patient reports that he or she is sick, the patient may be prompted to report particular symptoms (e.g., fever, nausea).

With respect to FIG. 36, an example of a Patient Message Page is shown, which may be included in a Patient Dashboard Page, Provider Dashboard Page, etc. As shown in FIG. 36, a Patient Message Page may be generated based on an initial message describing symptoms requiring a medical response. Further messages relating to discussion between the patient, patient's agent, or provider may then be automatically retrieved and displayed under the initial message that described symptoms requiring a medical response. FIG. 37 shows another example of Patient Message Page, except that the ability to send an additional message is further included.

With respect to FIG. 38, another example of a Medication Usage Page is shown.

With respect to FIG. 39, an example of a Patient Survey Page is shown. As shown in FIG. 39, a Patient Survey Page may be generated based on a message indicating to a computing device that a poll should be generated. For example, a message may be constructed in the following form: “(poll: Yes: No) Are you experiencing any chest pain?(/poll)”. A computing device may then send the message and any necessary instructions for displaying the poll. Responses to the poll may be received by a computing device and converted into a message or a summary. For example, a response to the poll shown in FIG. 39 may show the results of a patient's response (e.g., a filled-in circle for Yes or No). In some embodiments, a computing device may construct a summary or more based on two or more received messages relating to a poll (e.g., 30% of respondents selected “Yes”).

With respect to FIGS. 40-43, an example of a Message Template Page is shown. As shown in FIG. 40, a computing device may allow a provider to select a pre-existing template or create a custom message. For example, a provider may select a pre-existing template for flu shots. Further, a computing device may allow a provider to select the allowed method for sending of the message (e.g., email, text message, or both). Depending on the allowed method for sending of the message, a computing device may show a sample text message or an email message. A computing device may also allow a provider to edit the messages presented or to insert field codes. In some embodiments, a computing device may allow a provider to select patient responses to be included with the email, as shown in FIG. 40. In further embodiments, a computing device may allow providers to select the color of the response buttons or the computing device may automatically select colors for each response button.

As shown in FIG. 41, a preview of the email or text message may be shown to the provider by a computing device. As shown in FIG. 42, a computing device may allow a provider to select the recipients of the email or text message. For example, a user may select recipients based on gender, age range, current medication, first name, last name, full name, health conditions, medical history, vitals, appointments or procedures, or a period of time when their last visit occurred. In some embodiments, a user may select recipients based on other or additional criteria, such as symptoms, weight, patient behavior (e.g., smoking), preferred provider location, patient address, patient insurance, etc. As shown in FIG. 42, a computing device may then be instructed to perform a search based on the criteria. Based on the search results, a computing device may allow a provider to select all or some of the patients that fit the criteria. FIG. 43 shows an example of such search results. Once the provider is satisfied with the message and recipients, a computing device may then receive an instruction to send the message to the recipients.

With respect to FIG. 44, there is shown an embodiment of a methodology 4400 operable by a computing device for handling messages between a patient or a patient's agent and a provider. At step 4402, a computing device may receive a message. At step 4403, a computing device may then process the message according to instructions contained in Notification/Messaging Component 130. For example, if the message indicates a patient that is to receive a message, a computing device may retrieve information from Database Component 122 to determine the actual recipients (e.g., patient or patient's agents) and may also determine the allowed form of contact (e.g., email, text message, or both). As another example, if the message indicates that it contains the individual response to a group poll, Notification/Messaging Component 130 may contain instructions for the computing device to include such a response in a summary of the poll. At step 4404, a computing device may send the message to an intended recipient if the message is being sent instead of being received. At step 4406, a computing device may display the message, which may further include actions that may be taken with regard to the message (e.g., “Monitor”, “Archive”, “Reply”). In some embodiments, the computing device may also allow editing of the message, such as to include field codes. At step 4408, a computing device may receive the results of actions received by a user and store or forward them via Notification/Messaging Component 130.

With respect to FIG. 45, there is shown a methodology 4500 for selecting a pre-existing template or creating a custom message. At step 4502, a computing device may receive instructions to select a pre-existing template or create a custom message. At step 4504, if a pre-existing template is selected, the template may be retrieved, such as from Database Component 122. At step 4506, a computing device may receive instructions as to the message format to be used (e.g., email, text message, or both). At step 4508, a message field showing a text message may be displayed by the computing device, which may be blank or include information from a pre-existing template, wherein a blank message field may also be referred to as a blank template. At step 4510, a message field showing an email message may be displayed by the computing device, which may be blank or include information from a pre-existing template, wherein a blank message field may also be referred to as a blank template. At step 4512, a computing device may receive one or more edits to a message field, such as the text message field or email message field. At step 4514, a computing device may allow for the addition or removal of response buttons to be sent with the email message, and which may further allow for editing the content of response buttons. In some embodiments, the computing device may also allow for the selection of colors that should be used for the response buttons. At step 4516, a computing device may show a preview of how the text message or email message will appear to the recipient. At step 4518, a computing device may display criteria allowing for the search of potential recipients. At step 4520, a computing device may receive the selected criteria for searching of potential recipients and conduct a search for such recipients. At step 4522, a computing device may display a list of possible recipients that meet the selected search criteria. At step 4524, a computing device may receive a selection of the possible recipients (e.g., all or a subset thereof). At step 4526, a computing device may receive instructions to send the message based on the information provided in steps 4502-4524 and proceed to send the message.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures or may be omitted. For example, two blocks shown in succession may, in fact, be executed substantially concurrently, or the blocks may sometimes be executed in the reverse order, depending upon the functionality involved. It should also be noted that each block of the block diagrams or flowchart illustration, and combinations of blocks in the block diagrams and/or flowchart illustration, can be implemented by special purpose hardware-based systems that perform the specified functions or acts, or combinations of special purpose hardware and computer instructions. In addition, any references to steps of a methodology are used herein only to facilitate discussion, and carry no particular temporal or chronological significance unless otherwise indicated.

Those skilled in the art will appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings and descriptions provided herein.

What is claimed is:

1. A computer-implemented method for an enhanced management system to manage patient care and communication, the method comprising:
as implemented by one or more computer systems comprising computer hardware and memory, the one or more computer systems configured with specific executable instructions;
establishing an online message server for distributing messages, the online message server being accessible by devices communicating with the online message server over respective network communications and the online message server containing patient records in a patient database, wherein each patient record corresponds to a patient user and contains patient contact information, and agent records in an agent database, wherein each agent record corresponds to an agent user and contains agent contact information and agent permissions, wherein the agent permissions specify with respect to at least one patient record whether the agent user can receive messages sent to the patient user associated with the at least one patient record or send messages regarding the patient user associated with the at least one patient record;
from the online message server, enabling the devices to allow a patient user associated with a patient record to send an agent modification instruction to the online message server, wherein the agent modification instruction activates the online message server to enable or disable the agent permissions in one or more agent records associated with the patient user's patient record;
from the online message server, enabling the devices to generate a message, select a first set of recipients to receive the message based on the patient records contained in the patient database, and provide an instruction to send the message;
for each of the one or more first set of recipients, locating a set of agent records in the agent database that are associated with said recipient and determining whether to add an agent user to a second set of recipients based on the message and the agent permissions contained in said agent record, wherein the agent user is excluded from the second set of recipients if the agent permissions are disabled; and
sending the message to the first and second set of recipients.

2. The computer-implemented method of claim 1, wherein enabling the devices to generate a message further comprises the online message server being capable of:
providing a blank template or a list of pre-existing templates; and
receiving a filled in template or a selection of a pre-existing template from the list of pre-existing templates.

3. The computer-implemented method of claim 2, wherein enabling the devices to generate a message further comprises the online message server being capable of:
receiving an instruction to include one or more response options in the message and automatically generating such response options in the message.

4. The computer-implemented method of claim 3, wherein enabling the devices to generate a message further comprises the online message server being capable of:
generating the message in two or more formats suitable for sending by email, text, or other forms of communication.

5. The computer-implemented method of claim 4, wherein sending the message to the first and second set of recipients further comprises sending the message to each recipient in at least one format allowed by either the patient record or agent record associated with each recipient.

6. The computer-implemented method of claim 1, wherein from the online message server, the agent database is not accessible by the devices communicating with the online message server over respective network communications.

7. The computer-implemented method of claim 6, comprising the further step, from the online message server, of enabling the devices to receive the second set of recipients associated with the first set of recipients selected to receive the message.

8. A computer system comprising:
one or more hardware processors in communication with a computer readable medium storing software modules including instructions that are executable by the one or more hardware processors, the software modules including at least:
an online message server for distributing messages, the online message server being accessible by devices communicating with the online message server over respective network communications and the online message server containing patient records in a patient database, wherein each patient record corresponds to a patient user and contains patient contact information, and agent records in an agent database, wherein each agent record corresponds to an agent user and contains agent contact information and agent permissions, wherein the agent permissions specify with respect to at least one patient record whether the agent user can receive messages sent to the patient user associated with the at least one patient record or send messages regarding the patient user associated with the at least one patient record;
a first interface module residing on the online message server that enables the devices to allow a patient user associated with a patient record to send an agent modification instruction to the online message server, wherein the agent modification instruction activates the online message server to enable or disable the agent permissions in one or more agent records associated with the patient user's patient record;
a second interface module residing on the online message server that enables the devices to generate a message, select a first set of recipients to receive the message based on the patient records contained in the patient database, and provide an instruction to send the message;
for each of the one or more first set of recipients, locating a set of agent records in the agent database that are associated with said recipient and determining whether to add an agent user to a second set of recipients based on the message and the agent permissions contained in said agent record, wherein the agent user is excluded from the second set of recipients if the agent permissions are disabled; and
sending the message to the first and second set of recipients.

9. The system of claim 8, wherein a report relating to the management of a patient's care is generated by the online message server, and wherein the report correlates with established medical billing codes to generate a billing report.

10. The system of claim 8, wherein the agent permissions contained in each agent record are based on a legal document contained within each agent record.

11. The system of claim 8, wherein determining whether to add an agent user to a second set of recipients is further based on whether the message is of a type that can be received by each agent associated with each said agent record.
